SEQUENCE LISTING (38 sequences)

SEQ ID NO: 1 - DNA, 20 bp, Artificial Sequence
  Description of Artificial Sequence Synthetic primer
tcaagaacgaaagtcggagg

SEQ ID NO: 2 - DNA, 19 bp, Artificial Sequence
  Description of Artificial Sequence Synthetic primer
ggacatctaagggcatcac

SEQ ID NO: 3 - DNA, 20 bp, Artificial Sequence
  Description of Artificial Sequence Synthetic primer
ccctctcaccatctctgagc

SEQ ID NO: 4 - DNA, 20 bp, Artificial Sequence
  Description of Artificial Sequence Synthetic primer
cggttggggaacaagtagaa

SEQ ID NO: 5 - DNA, 20 bp, Artificial Sequence
  Description of Artificial Sequence Synthetic primer
gatatcggctccttcgatga

SEQ ID NO: 6 - DNA, 20 bp, Artificial Sequence
  Description of Artificial Sequence Synthetic primer
cagtccttccccatagcgta

SEQ ID NO: 7 - DNA, 20 bp, Artificial Sequence
  Description of Artificial Sequence Synthetic primer
gaaggttaagcgggaagagg

SEQ ID NO: 8 - DNA, 20 bp, Artificial Sequence
  Description of Artificial Sequence Synthetic primer
tccaggcgcttaaagttcat

SEQ ID NO: 9 - DNA, 21 bp, Artificial Sequence
  Description of Artificial Sequence Synthetic primer
gaagggcctctatgctaccca

SEQ ID NO: 10 - DNA, 18 bp, Artificial Sequence
  Description of Artificial Sequence Synthetic primer
tgggcgctggatacgaga

SEQ ID NO: 11 - DNA, 21 bp, Artificial Sequence
  Description of Artificial Sequence Synthetic primer
catcagccttgaccgctactg

SEQ ID NO: 12 - DNA, 21 bp, Artificial Sequence
  Description of Artificial Sequence Synthetic primer
cgcacgtagaccaggatcatg

SEQ ID NO: 13 - DNA, 17 bp, Artificial Sequence
  Description of Artificial Sequence Synthetic primer
ggcaagacttggtgcga

SEQ ID NO: 14 - DNA, 20 bp, Artificial Sequence
  Description of Artificial Sequence Synthetic primer
cgacgagatgacgtaccact

SEQ ID NO: 15 - DNA, 21 bp, Artificial Sequence
  Description of Artificial Sequence Synthetic primer
tcatcctcttcaccattttcg

SEQ ID NO: 16 - DNA, 21 bp, Artificial Sequence
  Description of Artificial Sequence Synthetic primer
ctggaagccaagatgtaccag

SEQ ID NO: 17 - DNA, 21 bp, Artificial Sequence
  Description of Artificial Sequence Synthetic primer
ctcatcatccctttctctctg

SEQ ID NO: 18 - DNA, 21 bp, Artificial Sequence
  Description of Artificial Sequence Synthetic primer
cctcttcatctccctcctctg

SEQ ID NO: 19 - DNA, 20 bp, Artificial Sequence
  Description of Artificial Sequence Synthetic primer
cctgcgcatctacctgatcg

SEQ ID NO: 20 - DNA, 20 bp, Artificial Sequence
  Description of Artificial Sequence Synthetic primer
aagctgaggccggagacact

SEQ ID NO: 21 - DNA, 18 bp, Artificial Sequence
  Description of Artificial Sequence Synthetic primer
ttcaccgtggtaggcaat

SEQ ID NO: 22 - DNA, 18 bp, Artificial Sequence
  Description of Artificial Sequence Synthetic primer
tttctcgctgagcgtacg

SEQ ID NO: 23 - DNA, 21 bp, Artificial Sequence
  Description of Artificial Sequence Synthetic primer
ccttcccgcctctcgtctctt

SEQ ID NO: 24 - DNA, 21 bp, Artificial Sequence
  Description of Artificial Sequence Synthetic primer
tctcgccgccgtcctcctctg

SEQ ID NO: 25 - DNA, 20 bp, Artificial Sequence
  Description of Artificial Sequence Synthetic primer
gtggtgatcgccgtgctgac

SEQ ID NO: 26 - DNA, 20 bp, Artificial Sequence
  Description of Artificial Sequence Synthetic primer
cgttttcggtagtcggggac

SEQ ID NO: 27 - DNA, 21 bp, Artificial Sequence
  Description of Artificial Sequence Synthetic primer
agccttggagaatggcagcac

SEQ ID NO: 28 - DNA, 21 bp, Artificial Sequence
  Description of Artificial Sequence Synthetic primer
ttcatcgtcctcctccagggc

SEQ ID NO: 29 - DNA, 21 bp, Artificial Sequence
  Description of Artificial Sequence Synthetic primer
agccttggagaatggcagcac

SEQ ID NO: 30 - DNA, 21 bp, Artificial Sequence
  Description of Artificial Sequence Synthetic primer
ttcatcgtcctcttccagggc

SEQ ID NO: 31 - DNA, 23 bp, Artificial Sequence
  Description of Artificial Sequence Synthetic primer
agcatgaggacacgaatctggct

SEQ ID NO: 32 - DNA, 23 bp, Artificial Sequence
  Description of Artificial Sequence Synthetic primer
ccgtcatcctcttcgtccttgtc

SEQ ID NO: 33 - DNA, 23 bp, Artificial Sequence
  Description of Artificial Sequence Synthetic primer
gtcaaagtgaagctggtggtgtc

SEQ ID NO: 34 - DNA, 23 bp, Artificial Sequence
  Description of Artificial Sequence Synthetic primer
ccatcatcctcttcgtccttgtc

SEQ ID NO: 35 - DNA, 23 bp, Artificial Sequence
  Description of Artificial Sequence Synthetic primer
gtcaaggtgaagctggtggtgtc

SEQ ID NO: 36 - DNA, 23 bp, Artificial Sequence
  Description of Artificial Sequence Synthetic primer
ataacccaagtaaggtaccctct

SEQ ID NO: 37 - DNA, 23 bp, Artificial Sequence
  Description of Artificial Sequence Synthetic primer
tacagggtcaaggtgaagctggt

SEQ ID NO: 38 - DNA, 23 bp, Artificial Sequence
  Description of Artificial Sequence Synthetic primer
ggtcatcacagtcgtcatccttc

(19) United States
(12) Patent Application Publication
(10) Pub. No.: US 2010/0048479 A1
(43) Pub. Date: Feb. 25, 2010

(54) ADRENAL GRK2 ACTIVITY AS A THERAPEUTIC TARGET FOR HEART FAILURE

(75) Inventors: Anastasios Lymperopoulos, Philadelphia, PA (US); Walter J. Koch, Broomall, PA (US)

Correspondence Address: DAVID S. RESNICK, NIXON PEABODY LLP, 100 SUMMER STREET, BOSTON, MA, 02110-2131, US

(73) Assignee: THOMAS JEFFERSON UNIVERSITY, Philadelphia, PA (US)

(21) Appl. No.: 12/513,713

(22) PCT Filed: Nov. 9, 2007
(86) PCT No.: PCT/US2007/023628

§ 371 Date: May 6, 2009

Related U.S. Application Data
(60) Provisional application No. 60/857,773, filed on Nov. 9, 2006.

(51) Int. Cl. A61K 38/16; A61K 31/711; A61K 31/7105; A61P 9/10

(52) U.S. Cl. 514/12; 514/44 R; 514/44 A

(57) ABSTRACT

The present invention relates to compositions and methods for the treatment of failing hearts. More specifically, the present invention provides for the inhibition of G-protein coupled receptor kinase 2 activity in the adrenal gland, which, for example, decreases catecholamine secretion and the sympathetic burden of the failing heart, thereby improving the cardiac adrenergic/inotropic reserve and overall contractile function.

### CROSS REFERENCE TO RELATED APPLICATION

This application claims benefit of U.S. provisional application No. 60/857,773 filed Nov. 9, 2006, the contents of which are incorporated herein by reference in the entirety.

### GOVERNMENT SUPPORT

The invention was made with Government support under Grant No. P01-HL075443-01 awarded by NIH/NHLBI. The Government has certain rights in the invention.

### BACKGROUND OF THE INVENTION

Despite recent advances in prevention and management of heart disease, death due to chronic heart failure continues to rise. Coronary heart disease kills more Americans than any other illness. The American Heart Association reports that nearly five million Americans live with chronic heart failure, and 550,000 new cases are diagnosed each year.

A salient feature of heart failure is elevated sympathetic nervous system (SNS) activity and outflow, indicated by increased circulating catecholamines. This increase reflects an adaptive process that compensates for decreased cardiac function through stimulation of β-adrenergic receptors (βARs). Yet as cardiac dysfunction continues, the compensatory increase of circulating catecholamine becomes maladaptive, contributing significantly to disease progression. In fact, levels of norepinephrine (NE) are associated with worsened prognosis in heart failure. Epinephrine (Epi) and, to a lesser extent, NE secretion from the adrenal medulla provide essentially all circulating catecholamines and is a fundamental component of SNS outflow. Chronic catecholamine elevation in the heart causes significant dysregulation of βARs and a myriad of molecular abnormalities including the up-regulation of the G-protein coupled receptor kinase 2 (GRK2 or βARK1), which contributes significantly to βAR and ventricular dysfunction. In addition, the increased circulating catecholamine and enhanced SNS activation lead to SNS-induced vasoconstriction of blood vessels that is known as sympatholysis.

Adrenergic signaling is also critical for normal SNS regulation and function primarily via α2-adrenergic receptors (α₂ARs). Studies have attributed almost all the presynaptic inhibitory autoreceptor function in the central SNS to α_{2A}AR, and this subtype is responsible for lowering sympathetic outflow and blood pressure in response to α₂AR-agonist drugs, like clonidine. Moreover, α2ARs are solely responsible for the autocrine feedback inhibition of catecholamine secretion from the adrenal gland induced by activation of nicotinic cholinergic receptors present on chromaffin cell membranes.

Insight into the role of sympatho-inhibitory α₂AR in regulation of cardiac function has come recently from studies of mice with genetic deletion of knockout of individual α₂ARs. Indeed, that α_{2A}AR- or α_{2C}AR knockout mice have significantly enhanced SNS activity and circulating catecholamine levels, with significantly worsened heart function after surgical pressure overload compared to similarly stressed control mice. Double α_{2A}/α_{2C} knockout mice exhibit an even worse cardiac phenotype and develop cardiomyopathy without stress. Of interest clinically, heart failure patients carrying a loss-of-function α_{2C}AR deletion mutation appear to have both worsened prognosis and increased risk to develop heart failure. Moreover, this mutation in a normal human population leads to increased SNS and adrenomedullary activity even at rest. Thus, α_{2A}Rs, including those in the adrenal gland, play a key role in regulation of SNS activity/outflow and of circulating catecholamine levels.

Despite this significance of α₂ARs in the SNS, their role in adrenal physiology, function, and catecholamine secretion in heart failure has not been investigated. Moreover, G-protein coupled receptor kinase expression and function in the adrenal gland are currently unknown, which could be critical because G-protein coupled receptor kinase 2 is increased in failing human hearts.

Therefore, there is a need for clinically relevant strategies for the regulation of adrenal ARs, circulating catecholamine, and enhanced SNS activation during heart failure. There is also a need for novel therapeutic sympatholytic strategies for treating heart failure.

### SUMMARY OF THE INVENTION

The present invention provides for the determination that G-protein coupled receptor kinase 2 (GRK2) up-regulation is the molecular mechanism for dysfunctional α₂AR signaling that can limit the clinical effectiveness of sympatholysis targeting this pathway. Moreover, GRK2 is responsible for enhanced adrenal secretion of catecholamines in heart failure, and inhibition of its activity in chromaffin cells can restore normal α₂AR-mediated control of SNS function and reduce catecholamine levels in heart failure. These results demonstrate adrenal GRK2 inhibition as a potential novel therapeutic sympatholytic strategy for heart failure.

In one embodiment, the invention provides a method for inhibiting both the down-regulating and desensitizing of peripheral α₂ARs in a target cell of a subject experiencing heart failure, comprising introducing to a target cell of a subject a GRK2 kinase inhibitor in an amount and under conditions such that α₂AR down-regulation and desensitization is lessened as a result of the decrease of adrenal GRK2 activity, thereby reducing the catecholamine release in said target cell of the subject.

Because circulating catecholamine also activates the circulatory renin-angiotensin-system and the endothelin system, and angiotensin II and endothelin are known to be harmful to the failing heart. Therefore, decreasing adrenal catecholamine secretion via adrenal GRK2 inhibition can help alleviate the detrimental effects of both renin-angiotensin-system (RAS) and endothelin (ET) systems in HF.

In one embodiment, the invention provides a method of treating heart failure in a subject by inhibiting the adrenal GRK2 activity. In one embodiment, inhibition is achieved by delivering to the subject a GRK2 antagonist. For example, the method may comprise administering to the adrenal gland of the subject a vector comprising a nucleic acid sequence encoding a GRK2 antagonist. The GRK2 inhibitor may be a βARKct peptide, a GRK2 siRNA, or a GRK2 antisense oligonucleotide. In a particular embodiment, the GRK2 antagonist is a βARKct peptide. The GRK2 kinase inhibitor can be administered to the subject by means of suprarenal or direct intra-suprarenal injection.

In another embodiment, the method involves contacting a target cell with the GRK2 inhibitor. The target cell of the subject experiencing a failing heart may a peripheral sympathetic neuron, a cardiac sympathetic terminal neuron or a chromaffin cell of the adrenal medulla. The GRK2 inhibitor may be a βARKct peptide, a GRK2 siRNA, or a GRK2 antisense oligonucleotide. The GRK2 kinase inhibitor can be administered to the target cell of the subject by means of suprarenal or direct intra-suprarenal injection.

The GRK2 inhibitor may be delivered to the subject either alone or in conjunction with other medicines such as beta-blockers, inhibitors of angiotensin-converting enzyme, or angiotensin receptor blockers.

Additionally, the approach provided for herein may prove a useful therapy for other diseases (endocrinological or not), that are also characterized by elevated SNS activity/outflow and catecholaminergic turnover, such as hyperthyroidism and pheochromocytoma (tumor of the adrenal medulla).

The features and details of the invention will become more apparent and appreciated by one skilled in the art to which this invention pertains from the following detailed description of the invention.

### DESCRIPTION OF THE DRAWINGS

FIG. 1A. Plasma epinephrine and norepinephrine levels in NLC or CSQ/Tg mice, sham or heart failure (HF) rats.*, p<0.05, #, p<0.01, n=5-7 mice/litter, *, p<0.05, n=5-6 rats/group.

FIG. 1B. Adrenal weight-to-body weight (AW/BW) ratios in NLC or CSQ/Tg mice, sham or HF rats. *, p<0.05, n=8-10 mice/litter or 6-7 rats/group.

FIG. 1C. Histograms of quantitative real-time (RT)-PCR of tyrosine hydroxylase (TH) gene on mRNA isolated from whole adrenal glands of NLC or CSQ/Tg mice, sham or HF rats.*, p<0.05, **, p<0.01, n=5 mice or rats/group.

FIG. 1D. Histograms of densitometric analyses of western blots of adrenal TH of NLC or CSQ/Tg mice, sham or HF rats. *, p<0.01, **, p<0.01, n=3 independent experiments.

FIG. 2A. Histograms of the quantitative RT-PCRs of adrenal α₂AR isoforms in NLC or CSQ/Tg mice, sham or HF rats.

FIG. 2B. Histograms of the α₂AR density in plasma membranes isolated from adrenal glands of NLC or CSQ/Tg mice, sham or HF rats. *, p<0.05, n=3 independent experiments.

FIG. 3A. Histograms of the quantitative real-time RT-PCRs of adrenal GRKs in CSQ/Tg or NLC mice, HF or sham rats. *, p<0.05, NS=Not significant at p<0.05, n=7 mice/litter or rats/group.

FIG. 3B. Histograms of densitometric analyses of western blots of adrenal GRK2 in CSQ/Tg or NLC mice, HF or sham rats. *, p<0.05.

FIG. 3C. Histograms of the quantitative real-time RT-PCR of adrenal βarr1 and βarr2 expression in NLC or CSQ/Tg mice, sham or HF rats. *, p<0.05.

FIG. 3D. Histogram of the quantitative real-time RT-PCRs and western blots for adrenal spinophilin in NLC or CSQ/Tg mice, sham or HF rats. *, p<0.05.

FIG. 4A. In vitro epinephrine and norepinephrine secretion from chromaffin cells isolated from adrenals of CSQ/Tg and NLC mice in response to nicotine treatment, following pre-treatment with vehicle (Nicotine) or with 10 μM UK14304 (UK14304+Nicotine). *, p<0.05.

FIG. 4B. In vitro epinephrine and norepinephrine secretion from chromaffin cells isolated from adrenals of HF and sham rats in response to nicotine treatment, following pre-treatment with vehicle (Nicotine) or with 10 μM UK14304 (UK14304+Nicotine). *, p<0.01; #, p<0.05.

FIG. 4C. In vitro catecholamine secretion from chromaffin cells isolated from adrenals of CSQ/Tg mice and transfected either with adenovirus encoding Green Fluorescent Protein alone (GFP) or with adenovirus encoding both βARKct and GFP (βARKct), and treated 24 hr post-transfection with nicotine, following pre-treatment either with Nicotine or with 10 μM UK14304 (UK14304+Nicotine)*, p<0.01; #, p<0.05.

FIG. 4D. In vitro catecholamine secretion from chromaffin cells isolated from adrenals of GFP or βARKct plus GFP (βARKct) transfected HF rats and treated 24 hr post-transfection with nicotine, following pre-treatment either with Nicotine or with 10M UK14304 (UK14304+Nicotine)*, p<0.05; #, p<0.05.

FIG. 4E. The average depolarization-induced Ca²⁺ entry in isolated HF or sham rat-derived chromaffin cells, after vehicle- or UK14304 (UK)-pretreatment. *, p<0.05, n=9-10 cells per condition.

FIG. 4F. Representative Ca²⁺ transient traces, measured as F₃₄₀/F₃₈₀ ratios, evoked by K⁺-induced depolarization of vehicle-pretreated (−UK) or UK14304-pretreated (+UK) sham rat-derived (♦) or HF rat-derived (▪) chromaffin cells.

FIG. 4G. Histogram of densitometric analyses of western blots of plasma membrane protein extracts prepared from rat primary chromaffin cells, transfected either with GFP or with βARKct, showing endogenous βarr1 membrane recruitment in response to treatment with UK14304 or vehicle. *, p<0.01.

FIG. 5A. Histograms of GRK2, tyrosine hydroxylase (TH), βarr1 total expression and membrane distribution in the adrenal glands of AdGFP or AdβARKct transfected rats. *P<0.05 vs. AdGFP; n=7 rats per group.

FIG. 5B. Histograms of GRK2 expression in cardiac extracts of AdGFP or AdβARKct transfected rats. *, p<0.05, compared to AdGFP, n=7 rat hearts/group.

FIG. 5C. Histograms of PAR density in the membrane of cardiac extracts of AdGFP or AdβARKct transfected rats. *, p<0.05, compared to GFP, n=7 rat hearts/group.

FIG. 5D. Ejection fraction (EF) of HF rats before and after gene delivery, as measured by echocardiography. *P<0.05 vs. AdβARKct before gene delivery or AdGFP after gene delivery; n=10 rats per group.

FIG. 5E. Basal and maximal dose of isoproterenol (Max. Iso)-stimulated +dP/dtₘₐₓ and −dP/dtₘᵢₙ responses of HF rats 7d after adrenal gene delivery. *P<0.05 vs. AdGFP-Max Iso; n=12 rats per group.

FIG. 5F. Plasma catecholamine levels 7d after adrenal gene delivery (AdGFP or AdβARKct) to HF rats, and after concomitant treatment with control vehicle (−Mox) or moxonidine (+Mox). *P<0.05 vs. AdGFP; **P<0.05 vs.−Mox; n=5 rats per group per treatment.

FIG. 6. Schematic representation of the pathophysiologic role of GRK2 and the therapeutic importance of its inhibition in heart failure.

FIG. 7A. Echocardiographic analysis of 3-month-old control NLC and CSQ/Tg mice. Left ventricular (LV) end-diastolic dimension (EDD). *, p<0.05, n=8 mice/litter.

FIG. 7B. Echocardiographic analysis of 3-month-old control NLC and CSQ/Tg mice. Ejection fraction. #, p<0.01, n=8 mice/litter.

FIG. 8A. Basal and isoproterenol-stimulated hemodynamic responses in 3-month-old NLC and CSQ/Tg mice showing +dP/dtₘₐₓ: first derivative of left ventricular (LV) pressure increase, *, p<0.01; n=7 mice/litter.

FIG. 8B. Basal and isoproterenol-stimulated hemodynamic responses in 3-month-old NLC and CSQ/Tg mice showing −dP/dtmin: first derivative of LV pressure decrease, *, p<0.01; n=7 mice/litter.

FIG. 8C. Basal and isoproterenol-stimulated hemodynamic responses in 3-month-old NLC and CSQ/Tg mice showing LVSPmax: Maximum LV systolic pressure. #, p<0.05; n=7 mice/litter.

FIG. 8D. Basal and isoproterenol-stimulated hemodynamic responses in 3-month-old NLC and CSQ/Tg mice showing LVEDP: LV end-diastolic pressure. **, p<0.05, n=7 mice/litter.

FIG. 9. Echocardiographic analysis of HF rats 10 weeks post-MI (HF) and age-matched sham-operated controls (sham) showing (A) ejection fraction; (B) LV EDD.*, p<0.05, n=10 rats/group.

FIG. 10. The retrograde suprarenal rat vein-mediated gene delivery technique. LSG: left suprarenal gland; RSG: right suprarenal gland; 1: inferior vena cava; 2: right renal vein; 3: left renal vein; 4: right suprarenal vein; 5: left suprarenal vein. Thick arrows: direction of blood flow; thin arrows: direction of adenoviral injection (retrograde delivery). Black-dotted circles: sites of the vein ligations preceding the virus injection.

FIG. 11. Plasma catecholamine levels in rats seven days after adrenal-specific in vivo gene delivery of AdGFP, AdGRK2, or AdβARKct. *, p<0.05, compared to AdGFP, A, p<0.05, compared to AdGFP, **, p<0.05, compared to AdGRK2, n=5 rats/group.

FIG. 12. In vitro epinephrine (FIG. 12A) and norepinephrine (FIG. 12B) secretion from chromaffin cells isolated from adrenals of HF rats infected in vivo with AdGFP, AdGRK2, or AdβARKct in response to 20 μM nicotine treatment, following pretreatment with vehicle (Nicotine) or with 10 μM UK14304 (UK+Nicotine). *, p<0.05, compared to AdGFP-UK+Nicotine, #, p<0.05 compared to AdβARKct-UK+Nicotine, n=9 rats.

### DETAILED DESCRIPTION

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages can mean±1%.

The inventions illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the inventions embodied therein herein disclosed can be resorted to by those skilled in the art, and that such modifications and variations are considered to be within the scope of this invention. Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Drawings are provided for illustration, not limitation.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

In addition, where features or aspects of the invention are described in terms of Markush groups, those skilled in the art will recognize that the invention is also thereby described in terms of any individual member or subgroup of members of the Markush group. Unless otherwise stated, the present embodiments were performed using standard procedures, as described, for example, in Maniatis et al., MOLECULAR CLONING: LAB. MANUAL (Cold Spring Harbor Lab., Cold Spring Harbor, N.Y., 1982); Sambrook et al., MOLECULAR CLONING: LAB. MANUAL (2 ed., Cold Spring Harbor Lab. Press, 1989); Davis et al., BASIC METHODS IN MOLECULAR BIO. (Elsevier Sci. Pub., Inc., NY, 1986); 152 METHODS IN ENZYMOLOGY: GUIDE TO MOLECULAR CLONING TECHNIQUES (Berger & Kimmerl, eds., Acad. Press Inc., San Diego, Calif., 1987); CURRENT PROTOCOLS IN MOLECULAR BIOLOGY (CPMB) (Ausubel et al., eds., John Wiley & Sons, Inc.); CURRENT PROTOCOLS IN PROTEIN SCI. (CPPS) or CURRENT PROTOCOLS IN IMMUNOL. (CPI) (latter two refs Coligan et. al., eds. John Wiley & Sons, Inc.).

The present invention relates to the discovery that G-protein receptor kinase 2 (GRK2), also known as β-adrenergic receptor kinase-1 (βARK1), up-regulation is the molecular mechanism for dysfunctional α₂AR signaling that limits the clinical effectiveness of sympatholysis targeting this pathway. Moreover, GRK2 is responsible for enhanced adrenal secretion of catecholamines in heart failure (HF), and the inhibition of GRK2 activity in chromaffin cells can restore normal α₂AR-mediated control of SNS function and reduce catecholamine levels in heart failure. The invention comprises adrenal GRK2 inhibition as a potential novel therapeutic sympatholytic strategy for HF.

A salient feature of HF is elevated SNS activity and outflow, reflected by increased circulating catecholamines. Initial catecholamine release in HF may be an adaptive process to compensate for decreased cardiac function through stimulation of β-adrenergic receptors (βARs). Cohn et al., 311 New Engl. J. Med. 819-23 (1984); Port & Bristow, 33 J. Mol. Cellular. Cardiology 887-905 (2001); Rockman et al., 415 Nature 206-12 (2002).

Catecholamines are hormones derived from the amino acid tyrosine-containing catechol and amine groups. Catecholamines are water soluble and bound to plasma proteins, so they circulate in the bloodstream. The most abundant catecholamines are epinephrine (adrenaline), norepinephrine (noradrenaline) and dopamine. Catecholamines are produced primarily by the chromaffin cells of the adrenal medulla, and to a lesser extent by the postganglionic fibers of the SNS. Epinephrine (Epi) and, to a lesser extent, norepinephrine (NE) secretion from the adrenal medulla provides essentially all circulating catecholamines and is a fundamental component of SNS outflow. They are released by the adrenal glands in situations of stress such as insufficient cardiac output, reduce blood circulation, psychological stress or low blood sugar levels.

As cardiac dysfunction continues, the compensatory increase of circulating catecholamine becomes maladaptive, however, and contributes significantly to disease progression. Rockman et al, 2002. In fact, higher levels of NE are associated with worsened prognosis in HF. Chronic catecholamine elevation in the heart causes significant dysregulation of βARs that include a myriad of molecular abnormalities. Id. Included is the up-regulation of GRK2, which contributes significantly to βAR and ventricular dysfunction. Hata et al., 37 J. Mol. & Cellular Cardiology 11-21 (2004).

Adrenergic signaling is also critical for normal SNS regulation and function primarily via α₂ARs. Studies have attributed almost all the presynaptic inhibitory autoreceptor function in the central SNS to α_{2A}AR, and this subtype is responsible for lowering sympathetic outflow and blood pressure in response to α₂AR-agonist drugs, like clonidine. Bylund et al., 46 Pharmacol. Rev. 121-36 (1994); Link et al., 273 Sci. 803-05 (1996); MacMillan et al., 273 Sci. 801-03 (1996). Moreover, α2ARs are solely responsible for the autocrine feedback inhibition of catecholamine secretion from the adrenal gland induced by activation of nicotinic cholinergic receptors present on chromaffin cell membranes. Young & Landsberg, Catecholamines and the adrenal medulla, in WILLIAMS TEXTBOOK OF ENDOCRINOLOGY 665-728 (9th ed., Foster, Kronenberg, Larsen, eds., 1998); Brede et al., 17 Mol. Endocrinol. 1640-46 (2003).

It has been reported that α_{2A}AR- or α_{2C}AR KO mice have significantly enhanced SNS activity and circulating catecholamine levels with significantly worsened heart function after surgical pressure overload compared to similarly stressed control mice. Brede et al., 106 Circulation, 2491-96 (2002). Double α_{2A}/α_{2C} KO mice exhibit an even worse cardiac phenotype and develop cardiomyopathy without stress. Hein et al., 402 Nature 181-84 (1999); Brum et al., 283H1 Am. J. Physiol. Heart Circ. Physiol. 838-45 (2002). Of interest clinically, HF patients carrying a loss-of-function α_{2C}AR deletion mutation appear to have worsened prognosis and increased risk to develop HF. Small et al., 347 New Engl. J. Med. 1135-42 (2002); Small et al., 43 Ann. Rev. Pharmacol. Toxicol. 381-411 (2003). Moreover, this mutation in a normal human population leads to increased SNS and adrenomedullary activity even at rest. Neumeister et al., 15 Pharmacogenetic Genomics 143-49 (2005). Thus, α_{2A}Rs, including those in the adrenal gland, play a key role in regulation of SNS activity/outflow and of circulating catecholamine levels.

Despite this significance of α₂ARs in the SNS, their role in adrenal physiology, function, and catecholamine secretion in heart failure have never been investigated. Moreover, GRK expression and function in the adrenal gland are currently unknown, which could be critical because GRK2 concentration is increased in failing human hearts. Rockman et al., 2002; Iaccarino et al., 26 Eur. Heart J. 1752-58 (2005).

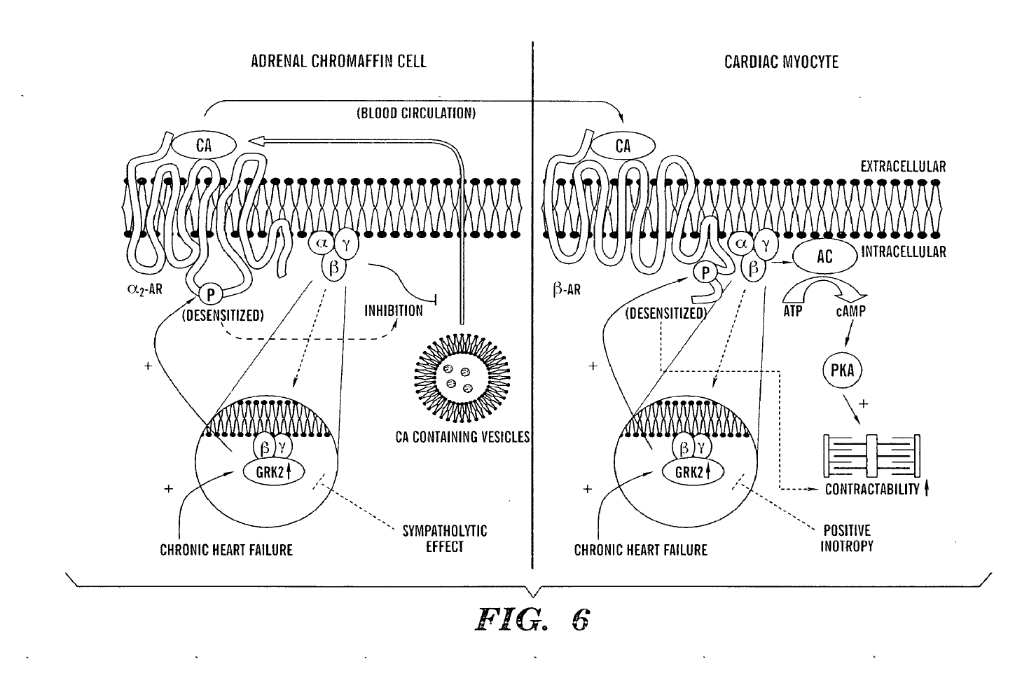

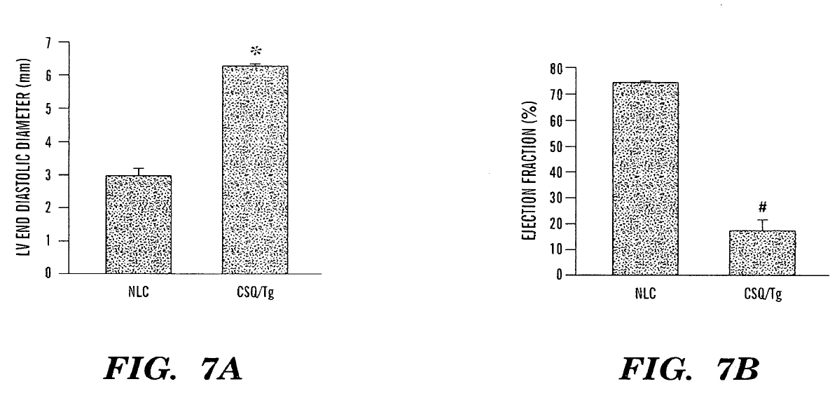

The α₂AR is the primary sympatho-inhibitory receptor in the SNS, and it was previously not known how this receptor was regulated in the peripheral SNS, including the adrenal gland. As shown here, in the normal adrenal gland several GRKs are expressed, and in HF GRK2 is selectively up-regulated, having a profound effect on α₂AR function and catecholamine release. The mechanism for this adrenal GRK2 up-regulation in HF is quite possibly due to enhanced central and adrenal SNS activity. The latter occurs in HF to stimulate the compromised heart, and circulating NE and Epi acting on adrenal α₂ARs could cause increased GRK2 desensitizing activity. Under a similar mechanism, it was shown previously that adrenergic activity via chronic stimulation of βARs in the heart is involved in cardiac GRK2 up-regulation. Iaccarino et al., 98 Circulation 1783-89 (1998); Iaccarino et al., 33 Hypertension 396-401 (1999). Moreover, GRK2 enhancement in the adrenal gland appears to be initially compensatory, but, as in the failing myocardium, continued up-regulation of GRK2 becomes maladaptive and contributes to adrenal pathology and SNS hyperactivity. Thus, based on the present discovery, as illustrated in FIG. 6, adrenal and myocardial GRK2 regulation in heart failure appears analogous with the difference being the primary adrenoceptor target: βARs in failing myocardium and α₂ARs in the failing adrenal gland.

Because circulating catecholamine also activates the circulatory renin-angiotensin-system and the endothelin system, and angiotensin II and endothelin are known to be harmful to the failing heart. Therefore, decreasing adrenal catecholamine secretion via adrenal GRK2 inhibition can help alleviate the detrimental effects of both renin-angiotensin-system (RAS) and endothelin (ET) systems in HF.

Previous work identified enhanced myocardial GRK2 activity as a key target for HF pathogenesis, as inhibition of GRK2 in the heart appears to be a beneficial therapeutic strategy. Rockman, 415 Nature 206-12, 2002; Hata et al., 37 J. Cell. Cardiol. 11-21 (2004). With the data presented herein, showing GRK2 to be a critical regulator of adrenal catecholamine secretion, enhanced adrenal GRK2 activity in HF appears to also be an attractive therapeutic target. This is particularly important because the data presented herein shows that GRK2 inhibition, via βARKct expression in chromaffin cells isolated from HF animals and in adrenal glands of HF rats in vivo, reverses α₂AR dysfunction and allows these receptors to effectively regulate and lower catecholamine secretion, which in turn significantly improves cardiac function and inotropic responsiveness to catecholamine stimulation. The βARKct clearly reversed α₂AR desensitization as demonstrated by increased signaling and lower βarr1 membrane translocation in chromaffin cells, and also by decreasing adrenal GRK2 and TH expression and adrenal βarr1 membrane translocation in vivo. In addition, in vivo inhibition of adrenal GRK2 by βARKct normalizes circulating catecholamine levels in HF and improves inotropic responses to sympathetic stimulation by up-regulating αARs and decreasing GRK2 expression/activity of the chronically failing heart. Thus, these results reveal that GRK2 does exert significant control on α₂AR signaling and function in the adrenal gland and further strengthens the conclusion that the βARKct, via its inhibition of GRK2 activity, can have profound effects in vivo on GPCR signaling.

FIG. 6 shows the schematic representation of the pathophysiologic role of GRK2 and the therapeutic importance of its inhibition in HF. Chronic HF leads to up-regulation of GRK2, both in cardiac myocytes and in adrenal chromaffin cells. In the cardiac myocyte, this results in increased phosphorylation and desensitization (inset) of βARs, which abrogates the CA-induced signaling of these receptors, through Gs protein-AC-PKA, to increased contractility. Thus, in chronic HF, the increased amounts of circulating (or locally released) CA's, present at cardiac βARs, are unable to elicit the pro-contractile response of these receptors, hence cardiac inotropic reserve diminishes and the “vicious circle” of sympathetic overstimulation of the failing heart ensues and/or is perpetuated. In the adrenal chromaffin cell, increased GRK2 results in increased phosphorylation and desensitization (inset) of α2ARs, which diminishes the CA-induced, Gi/Go protein-mediated signaling of these receptors to inhibition of catecholamine secretion, leading, over time, to accumulatively increasing amounts of circulating CA's in chronic heart failure. Therefore, simultaneous inhibition of GRK2 in the heart and in the adrenal gland, e.g. with a systemically delivered pharmacological inhibitor, has dual therapeutic efficacy in chronic HF, as it restores both cardiac inotropic reserve (positive inotropy in the myocardium) and cardiac sympathetic stimulation (sympatholytic activity in the adrenal gland) to normal. CA: Catecholamine, AC: Adenylyl cyclase, ATP: Adenosine triphosphate, cAMP: cyclic Adenosine monophosphate, PKA: Protein Kinase A.

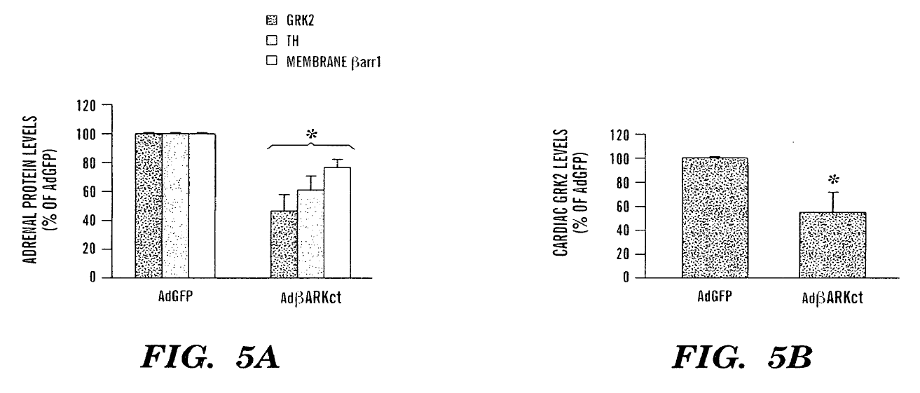

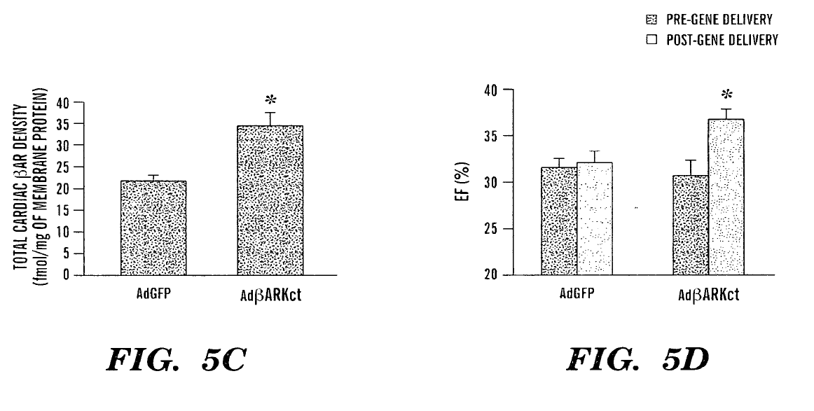

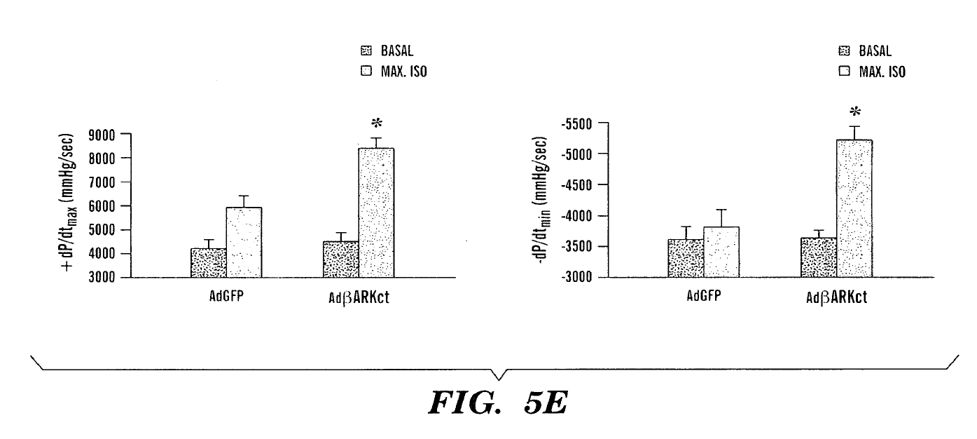

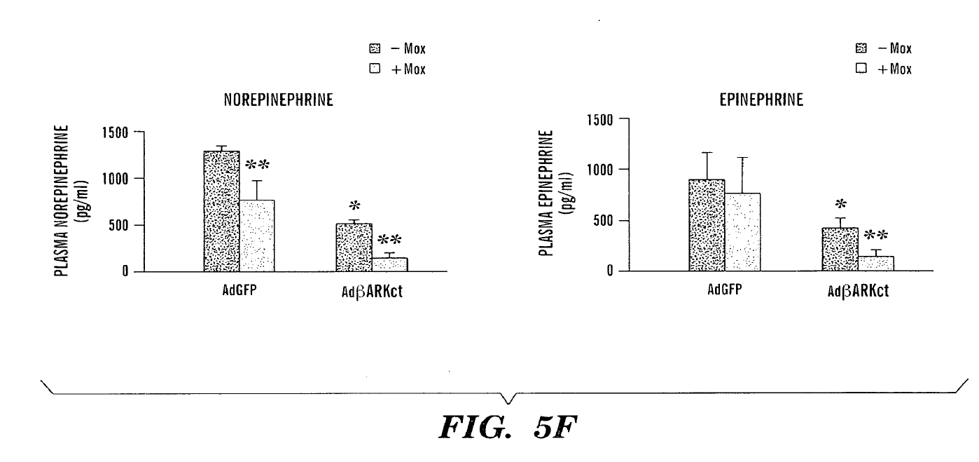

GRK2 data from the adrenal gland presented herein can also mean that GRK2-mediated α₂AR desensitization/down-regulation in chronic HF is in operation in peripheral sympathetic neurons as well, including cardiac sympathetic nerve terminals. Interestingly, because cardiac GRK2 inhibition has shown to rescue several animal models of HF (Rockman et al., 2002; Hata et al., 2004), and be synergistic with βAR blockade (Harding et al., 98 P.N.A.S. USA 5809-14 (2001)), the use of a small molecule GRK2 inhibitor can actually be beneficial due to both its effect on cardiac function in the myocardium and to lowering catacholamine secretion at the level of the adrenal gland (FIG. 6). Moreover, sympatholysis via GRK2 inhibition in the adrenal gland can have clear advantages over existing strategies including not only sparing the myocardium of the noxious effects of catecholamines as β blockers currently do, but by decreasing circulating catecholamine levels, peripheral toxicities elicited by these hormones would be decreased. Included in these would be the amelioration of chronic activation of the renin-angiotensin and endothelin systems, which are known to be activated by circulating catecholamines in heart failure, and which can play a detrimental role in heart failure. Floras, 195 Circulation 1753-55 (2002). Thus, inhibition of adrenal GRK2 would be synergistic with cardiac αAR blockade in heart failure, as inhibition of myocardial GRK2 activity is. Harding et al., 2001. Moreover, it shown herein that βARKct expression in the heart of HF mice (CSQ/Tg) and subsequent improvement of cardiac function does appear to feedback also to lower SNS activity and circulating catecholamine levels, which is an additional important and novel finding of this study (FIG. 5F). These findings do indicate that even outside of the βARKct's action in the myocardium, there is potential for synergy with β-blockers in HF and advantages mentioned above. Therefore, GRK2 inhibition in heart failure has broader therapeutic implications and the present specification provides new evidence that targeting this kinase, even outside of the failing myocardium, can be of therapeutic value.

One important currently applicable clinical translation for the present invention is the fact that GRK2-mediated α₂AR desensitization/down-regulation observed in heart failure could be mechanistically responsible for the poor sympatho-inhibitory efficacy of α₂AR-agonist drugs, such as clonidine and moxonidine, in heart failure. These drugs were posited to be potentially effective in heart failure to combat the sympathetic over-activation of the failing heart, however, since peripheral α₂ARs, such as in the adrenal medulla, and possibly in other peripheral sympathetic neuronal systems including the cardiac sympathetic nerve terminals, are desensitized and down-regulated in heart failure, the effectiveness of these agents would be expected to be severely diminished. Indeed, down-regulation of peripheral (and not central) α2ARs, and diminished sympatholytic responses to clonidine administration in HF animal models, as well as in human heart failure patients, have already been reported. Aggarwal et al., 37 J. Am. Coll. Cardiol. 1246-51 (2001); Lang et al., 30 Hypertension 392-97 (1997).

In addition, two recent large clinical trials of the sympatholytic drug moxonidine in heart failure, MOXSE and MOXCON, showed that significant reduction of SNS activity was achieved by the drug, (Swedberg et al., 105 Circulation 1797-1803 (2002); Swedberg et al., 35 J. Am. Coll. Cardiol. 398-404 (2002)), thus validating the concept of sympatholytic agents as a therapeutic modality in heart failure. These trials were terminated early, however, due to excessive adverse effects and fatalities. Floras, 2002; Swedberg et al., 2002. Therefore, one of the several possible reasons that might have accounted for the failure of these trials, apart from their poor design (Floras, 2002), could very well be the diminished function of peripheral sympatho-inhibitory α₂ARs due to elevated GRK2-mediated desensitization/down-regulation in heart failure, as present study reports for the adrenal gland. In fact, since central α₂ARs seem to function properly, and only peripheral α₂ARs appear to function inadequately in human heart failure (Aggarwal, 2001), α₂AR-agonists, such as moxonidine, would be expected to exert some degree of sympatholysis in heart failure, as indeed observed in the MOXSE and MOXCON trials, but this sympatholytic effect might not suffice or might be exerted in the wrong place, i.e. in parts of SNS other than the ones in which it should occur, i.e. cardiac sympathetic nerve terminals and the adrenal glands. Therefore, these key peripheral components of SNS might be left unaffected by α₂AR-agonist drugs because of the blunted and inappropriate α₂AR function of these tissues in heart failure. Therefore, the results presented herein provide for the first time a molecular mechanism for the observed peripheral α₂AR down-regulation and decreased clinical efficacy of α₂AR-agonist drugs in heart failure. Moreover, they suggest that GRK2 inhibition might circumvent these therapeutic efficacy obstacles that α₂AR-agonists and other similar sympatholytic drugs face in heart failure.

A shown schematically in FIG. 6, the present invention indicates that GRK2 inhibition can act as a dual therapeutic modality in chronic HF, depending on the target organ: although inhibition of this kinase in the heart provides a powerful mechanism for enhancing the function of the failing myocardium (Rockman et al., 2002; Hata et al., 2004), inhibition of this kinase in the adrenal gland (and possibly in other peripheral sympathetic neurons), provides a novel sympatholytic therapy to counteract the chronic deleterious sympathetic overstimulation of the failing heart by catecholamines and to improve its inotropic reserve and responsiveness to catecholamines. Moreover, this sympatholytic therapy would be efficacious in chronic heart failure, in contrast to α₂AR-agonist treatment, and it could possibly lack many of the side-effects of the latter agents, which, quite frequently, preclude their use in therapeutic practice. Guyenet et al., 273 Am. J. Physiol. R1580-84 (1997). Therefore, pharmacological GRK2 inhibitors (or targeted βARKct gene therapy) should be pursued for treatment of chronic heart failure, as they could help restore the cardio-toxic pharmacodynamic and bioenergetic imbalance between SNS hyperactivity and the chronically failing heart.

Hence, in one embodiment, the invention provides a method of treating heart failure in by inhibiting the adrenal GRK2 activity. The method may comprise administering to the adrenal gland of the subject a GRK2 antagonist. As used herein, the term GRK2 antagonist refers to a molecule that inhibits the activity of GRK2 or the expression of GRK2. For example, the GRK2 antagonist may be a βARKct peptide.

In another embodiment of the invention, the method may comprise administering to the adrenal gland of the subject a vector comprising a nucleic acid sequence that encodes a GRK2 antagonist. For example, the GRK2 antagonist may be a βARKct peptide

More specifically, βARK1ct is a peptide inhibitor of GRK2 activity. The peptide inhibitor corresponds to the carboxy-terminal 194 amino acids of GRK2 (corresponding to the amino acid residues 495-689 of mouse GRK2), and contains the binding domain for the βγ-subunits of G proteins (G_{βγ}), which is required for GRK2 membrane translocation and activation (Koch et al., 268 Science 1350-53 (1995); Pitcher et al., 257 Science 1264-67 (1992); Kock et al., 269 J. Biol. Chem. 6193-97 (1994). The βARKct competes with endogenous GRK2 for G_{βγ} binding, thereby inhibiting GRK2 activity. Akhter et al., 100 Circulation 648-53 (1999). The Genbank Accession numbers of the Homo sapiens adrenergic, beta, receptor kinase 1 are NM_{—}001619, BC037963, BC090863, M80776, and X61157.

In one embodiment, the coding nucleic acid sequence of βARK1ct can be PCR cloned into expression vectors by methods known in the art. The term “vector” used herein may mean a nucleic acid sequence containing an origin of replication and gene expression elements such as protomor and enhancer regions. A vector may be a plasmid, bacteriophage, episome, bacterial artificial chromosome, viral vector, or yeast artificial chromosome. A vector may be a DNA or RNA vector. A vector may be either a self replicating extra-chromosomal vector or a vector that integrates into a host genome. The term “vectors” used interchangeably with “plasmid” refer to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other forms of expression vectors known by those skilled in the art which serve the equivalent functions can also be used. Expression vectors comprise expression vectors for stable or transient expression of the encoding DNA.

In a particular embodiment, the expression vector carrying the coding sequence of βARK1ct is a viral vector, for example, adeno-associated virus, an adenovirus, a lentivirus, or a retrovirus. Such viral vectors form favorable vectors as they function as gene delivery vehicle as well as expression vectors, delivering the gene of interest into cells for gene expression. Cloning and production of an adenovirus expressing βARK1ct is described in detail in Drazner et al., 99 J. Clin. Invest. 288-96 (1997).

A simplified system for generating recombinant adenoviruses is presented by He et. al., 95 P.N.A.S. USA 2509-14 (1998). The appropriate nucleic acid segments can be made in a shuttle vector, e.g. pAdTrack-CMV. The resultant plasmid may be linearized by digesting with restriction endonuclease Pme I, and subsequently co-transformed into E. coli BJ5183 cells with an adenoviral backbone plasmid, e.g. pAdEasy-1 of Stratagene's AdEasy™ Adenoviral Vector System. Recombinant adenovirus vectors may be selected for kanamycin resistance, and recombination confirmed by restriction endonuclease analyses. Finally, the linearized recombinant plasmid may be transfected into adenovirus packaging cell lines, for example HEK 293 cells (E1-transformed human embryonic kidney cells) or 911 (E1-transformed human embryonic retinal cells). Fallaux et al., 7 Human Gene Therapy 215-22 (1996). Recombinant adenovirus may be generated within the HEK 293 cells.

In another embodiment, a recombinant lentivirus can be used for the delivery and expression of the βARK1 ct in adrenal cells. The HIV-1 based lentivirus may effectively transduce a broader host range than the Moloney Leukemia Virus (MoMLV)-base retroviral systems. Preparation of the recombinant lentivirus can be achieved using, e.g., the pLenti4/V5-DEST™, pLenti6/V5-DES™ or pLenti vectors together with ViraPower™ Lentiviral Expression systems from Invitrogen.

In yet another embodiment, a recombinant adeno-associated virus (rAAV) may be used for the expression of the βARK1ct in adrenal cells. Because AAV is non-pathogenic and does not illicit an immune response, a multitude of pre-clinical studies have reported excellent safety profiles. The rAAVs are capable of transducing a broad range of cell types and transduction is not dependent on active host cell division. High titers, >10⁸ viral particle/ml, are obtained easily in the supernatant and 10¹¹ viral particle/ml to 10¹² viral particle/ml with further concentration. The transgene is integrated into the host genome, providing long term and stable expression.

Large scale preparation of AAV vectors may be made, for example, by a three-plasmid co-transfection of a packaging cell line: AAV vector carrying the chimeric DNA coding sequence, AAV RC vector containing AAV rep and cap genes, and adenovirus helper plasmid pDF6, into 50 mm×150 mm plates of sub confluent 293 cells. Cells may be harvested three days after transfection, and viruses are released by three freeze-thaw cycles or by sonication.

AAV vectors may then be purified by two different methods depending on the serotype of the vector. AAV2 vector is purified by the single-step gravity-flow column purification method based on its affinity for heparin. Auricchio et al., 12 Human Gene Therapy 71-76 (2001); Summerford & Samulski, 72 J. Virol. 1438-45 (1998); Summerford & Samulski, 5 Nat. Med. 587-88 (1999). AAV2/1 and AAV2/5 vectors may be purified by three sequential CsCl gradients.

In one embodiment, the expression of βARK1ct is driven by an adrenal specific promoter, for example, DAX-1 promoter. See U.S. Pat. No. 6,465,627.

In one embodiment, the GRK2 up-regulation may be counteracted by a GRK2 siRNA, GRK2 shRNA or GSK2 anti-sense oligonucleotide. GRK2 siRNA, GRK2 shRNA and GSK2 anti-sense oligonucleotide function to interfere with the translation of the GRK2 mRNA. In one embodiment, RNA interference techniques can be used to reduce the expression of GRK2 in the adrenal glands. Other RNA interfering molecules include but are no limited to short-temporal RNA (stRNA), small interfering RNA (siRNA), short-hairpin RNA (shRNA), microRNA (miRNA), double-stranded RNA (dsRNA). See, e.g., Baulcombe, 297 Science 2002-03 (2002). The dsRNA molecules, e.g. siRNA, may also contain 3′ overhangs, such as 3′UU or 3′TT overhangs. In one embodiment, the siRNA molecules of the present invention do not include RNA molecules that comprise ssRNA greater than about 30 bases to 40 bases, about 40 bases to 50 bases, about 50 bases or more. For example, the siRNA molecules of the present invention may have a double stranded structure. More particularly, the siRNA molecules of the present invention may be double stranded for more than about 25%, more than about 50%, more than about 60%, more than about 70%, more than about 80%, or more than about 90% of their length.

As used herein, RNA interference (also called “RNA-mediated interference” abbreviated RNAi) refers to a mechanism for RNA-guided regulation of gene expression in which double-stranded ribonucleic acid inhibits the expression of genes with complementary nucleotide sequences. Conserved in most eukaryotic organisms, the RNAi pathway is thought to have evolved as a form of innate immunity against viruses and also plays a major role in regulating development and genome maintenance.

The RNAi pathway is initiated by the enzyme dicer, which cleaves double-stranded RNA (dsRNA) to short double-stranded fragments of 20 base pairs to 25 base pairs. One of the two strands of each fragment, known as the guide strand, is then incorporated into the RNA-induced silencing complex (RISC) and base-pairs with complementary sequences. A well-studied outcome of this recognition event is a form of post-transcriptional gene silencing. This occurs when the guide strand base pairs with a mRNA molecule and induces degradation of the mRNA by argonaute, the catalytic component of the RISC complex. The short RNA fragments are known as small interfering RNA (siRNA) which are perfectly complementary to the gene they are suppressing, as they are derived from long dsRNA of that same gene; or MicroRNA (miRNA) which are derived from the intragenic regions (or an intron) and are thus only partially complementary. The RNAi pathway has been particularly well-studied in certain model organisms such as the nematode worm Caenorhabditis elegans, the fruit fly Drosophila melanogaster, and the flowering plant Arabidopsis thaliana.

Numerous specific siRNA molecules have been designed that have been shown to inhibit gene expression. Ratcliff et al., 276 Science 1558-60 (1997); Waterhouse et al., 411 Nature 834-42 (2001). In addition, specific siRNA molecules have been shown to inhibit, for example, HIV-1 entry to a cell by targeting the host CD4 protein expression in target cells thereby reducing the entry sites for HIV-1 which targets cells expressing CD4. Novina et al., 8 Nature Medicine 681-86 (2002). Short interfering RNA have further been designed and successfully used to silence expression of Fas to reduce Fas-mediated apoptosis in vivo. Song et al., 9 Nature Medicine 347-51 (2003).

As used herein a “siRNA” refers to a nucleic acid that forms a double stranded RNA, which double stranded RNA has the ability to reduce or inhibit expression of a gene or target gene when the siRNA is expressed in the same cell as the gene or target gene. The double stranded RNA siRNA can be formed by the complementary strands. The complementary portions of the siRNA that hybridize to form the double stranded molecule typically have substantial or complete identity. In one embodiment, a siRNA refers to a nucleic acid that has substantial or complete identity to a target gene and forms a double stranded siRNA. The sequence of the siRNA can correspond to the full length target gene, or a subsequence thereof. Typically, the siRNA is at least about 15 nucleotides to 50 nucleotides in length (e.g., each complementary sequence of the double stranded siRNA is about 15 nucleotides to 50 nucleotides in length, and the double stranded siRNA is about 15 base pairs to 50 base pairs in length, for example about 19 to 30 base nucleotides, such as about 20 nucleotides to 25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19-nucleotides to 25-nucleotides, anti-sense strand, followed by a nucleotide loop of about 5-nucleotides to 9-nucleotides, and the analogous sense strand. Alternatively, the sense strand may precede the nucleotide loop structure and the anti-sense strand may follow.

Small interfering RNAs to GSK2 can be designed by one skilled in the art, using web-based programs. For example, siDirect is a web-based online software system for computing highly effective siRNA sequences with maximum target-specificity for mammalian RNA interference (RNAi). Naito et. al., 23 Nucleic Acid Res. WI 24-9 (2004). Other web-based algorithms for designing siRNA include but are not limited to SDS by siRNA Design Software, siRNA DNA Designer, and Rational siRNA Design Template (Reynolds et al., (2004), 22 Nature Biotech. 326-30 (2004)), and siRNA Wizard™ from InvivoGen.

While there must be some degree of sequence identity with the target gene, it is also known that the RNA interference molecules does not have to match perfectly to its target sequence. For example, the 5′ and middle part of the anti-sense (guide) strand of the siRNA is perfectly complementary to the target nucleic acid sequence.

The RNA interference molecules according to the present invention may be produced using any known techniques such as direct chemical synthesis. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs. Commercial vendors include QIAGEN Inc. (Valencia, Calif.) and AMBION Inc. (Austin, Tex.). Examples of methods of preparing such RNA interference are shown, for example in an International Patent applications No. PCT/US03/34424 and No. PCT/US03/34686.

In one embodiment, the siRNAs can be PCR cloned into expression vectors by methods known in the art. Examples of vectors for cloning siRNAs include but are not limited to siRNA cloning vector (pGB) from AbCam Inc., pSUPER or pSUPERretro vectors, and siRNA silencing vectors from Ambion Inc.

In one embodiment, the liposome encapsulate the βARK1 expression vector or siRNA for delivery into to adrenal cells. In one embodiment, the βARK1 expression vector or siRNA are condensed with a cationic polymer, e.g., PEI, polyamine spermidine, and spermine, or a cationic peptide, e.g., protamine and poly-lysine, and encapsulated in the lipid particle. The liposomes can comprise multiple layers assembled in a step-wise fashion.

Lipid materials well known and routinely utilized in the art to produce liposomes. Lipids may include relatively rigid varieties, such as sphingomyelin, or fluid types, such as phospholipids having unsaturated acyl chains. “Phospholipid” refers to any one phospholipid or combination of phospholipids capable of forming liposomes. Phosphatidylcholines (PC), including those obtained from egg, soy beans or other plant sources or those that are partially or wholly synthetic, or of variable lipid chain length and unsaturation are suitable for use in the present invention. Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is a negatively-charged lipid often used in formulations.

Other suitable phospholipids include phosphatidylethanolamines, phosphatidylinositols, sphingomyelins, and phosphatidic acids containing lauric, myristic, stearoyl, and palmitic acid chains. For the purpose of stabilizing the lipid membrane, one may add an additional lipid component, such as cholesterol. Lipids for producing liposomes according to the invention may include phosphatidylethanolamine (PE) and phosphatidylcholine (PC) in further combination with cholesterol (CH). For example, a combination of lipids and cholesterol for producing the liposomes of the invention comprise a PE:PC:Chol molar ratio of 3:1:1. Further, incorporation of polyethylene glycol (PEG) containing phospholipids is also contemplated by the present invention.

In addition, in order to prevent the uptake of the liposomes into the cellular endothelial systems and enhance the uptake of the liposomes into the tissue of interest, the outer surface of the liposomes may be modified with a long-circulating agent. The modification of the liposomes with a hydrophilic polymer as the long-circulating agent is known to enable to prolong the half-life of the liposomes in the blood

Liposomes encapsulating the nucleic acid segments described herein can be obtained by any method known to the skilled artisan. For example, the liposome preparation of the present invention can be produced by reverse phase evaporation (REV) method (see U.S. Pat. No. 4,235,871), infusion procedures, or detergent dilution. A review of these and other methods for producing liposomes may be found in 1 LIPOSOMES, (Marc Ostro, ed., Marcel Dekker, Inc., NY, 1983). See also Szoka et al., 9 Ann. Rev. Biophys. Bioeng. 467 (1980).

In one embodiment, the invention provides a method for inhibiting the down-regulation and desensitization of peripheral α₂-adrenergic receptors (α₂-ARs) in a target cell of a subject experiencing HF, by introducing into a target cell of a subject a GRK2 kinase inhibitor in an amount and under conditions such that α₂-AR down-regulation and desensitization is lessened, as a result of the decrease of adrenal GRK2 activity, thereby reducing the catecholamine release in said target cell of the subject. The target cell of the subject experiencing a failing heart may a peripheral sympathetic neuron, a cardiac sympathetic terminal neuron or a chromaffin cell of the adrenal medulla. The GRK2 kinase inhibitor is a βARKct peptide, a GRK2 siRNA, or a GRK2 antisense oligonucleotide. The GRK2 kinase inhibitor can be administered to the target cell of the subject by means of suprarenal or direct intra-suprarenal injection.

Adrenal GRK2 inhibition can provide several advantages over existing heart failure pharmacotherapies. For example, it provides for novel efficacious sympatholytic therapy, which is needed for the treatment of chronic heart failure, because this devastating disease is characterized and reinforced by the elevated Sympathetic Nervous System (SNS) activity/outflow. Additionally, the approach provided for herein targets a non-cardiac tissue, so cardiovascular adverse effects can be expected to be minimal. Furthermore, the instant approach can reduce the sympathetic burden of the failing heart, which beta-blockers are prescribed to counteract, therefore it can synergize with beta-blockers, allowing for decrease in dosage prescribed for heart failure. Hence, it can help reduce these drugs' adverse effects, including the risk for further deterioration of cardiac contractility.

Also, the present approach can reduce the activation of circulatory RAS and ET, therefore decreasing the amount and detrimental effects of Angiotensin II (and ET) on the failing heart, such as cardiac maladaptive hypertrophy and fibrosis. Thus, it can act in synergism with inhibitors of angiotensin-converting enzyme (ACE inhibitors) and Angiotensin Receptor Blockers (ARBs) in heart failure therapy. Moreover, chronic concomitant administration of adrenal GRK2 inhibitor plus beta-blocker therapy can better, and bioenergetically and pharmacodynamically, and more safely preserve cardiac reserve and catecholaminergenic responsiveness in chronic heart failure than beta-blockers alone. Finally, it can demonstrate a therapeutically very important synergism with cardiac GRK2 inhibition, as it normalizes the cardiotoxic increase of the sympathetic input to the failing heart, while the latter improves the inotropy (the force with which the heart muscle contracts) of the failing heart.

Hence, in one embodiment, the invention described herein provides for a novel therapy for chronic heart failure, either alone or in conjunction with beta-blockers, ACE inhibitors, or ARBs. Additionally, the invention herein can prove a useful therapy for other diseases (endocrinological or not), that are also characterized by elevated SNS activity/outflow and catecholaminergic turnover, such as hyperthyroidism and pheochromocytoma (tumor of the adrenal medulla).

In one embodiment, the invention provides a method for treating an endocrinological or a non-endocrinological disease characterized by an elevation of (SNS) activity/outflow and catecholamine turnover in a subject comprising introducing into a target cell of the subject a therapeutically effective amount of a GRK2 kinase inhibitor and a pharmaceutically acceptable carrier.

The term “therapeutically effective amount” refers to an amount that is sufficient to effect a therapeutically significant reduction in endocrinological or a non-endocrinological disease symptoms as well as slow the progression of disease over time. The term also refers to that amount necessary to attain, at least partly, the desired effect, of reducing, ameliorating, stopping, abating, alleviating, and inhibiting the symptoms associated with the endocrinological or a non-endocrinological disease, and also control and prevent further progression of the disease. Such amounts will depend, of course, the severity of the condition and individual patient parameters including age, physical condition, size, weight and concurrent treatment. These factors are well known to those of ordinary skill in the art and can be addressed with no more than routine experimentation. Generally, a maximum dose be used, that is, the highest safe dose according to sound medical judgment. It will be understood by those of ordinary skill in the art; however, that a lower dose or tolerable dose may be administered for medical reasons, psychological reasons or for virtually any other reason.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

In one embodiment, the invention described herein also provides a pharmaceutical composition for treating or preventing chronic HF in a subject comprising a therapeutically effective amount of a GRK2 kinase inhibitor suitable for delivery to adrenal cells, wherein the treatment lessens down-regulation and desensitization of α₂-adrenergic receptors in adrenal cells of the subject experiencing HF, which results from the decrease of adrenal GRK2 activity, thereby reducing the catecholamine release in the adrenal cells of the subject.

In another embodiment, the invention described herein provides a combined pharmaceutical composition for treating or preventing chronic heart failure in a subject comprising a GRK2 kinase inhibitor and an agent selected from the group consisting of beta-blocker, ACE inhibitor, or ARB, wherein delivery of said pharmaceutical results in down-regulation and desensitization of α₂-adrenergic receptors in a cell of a subject experiencing chronic HF.

The beta-blocker of the combined pharmaceutical composition may be one or more of Sectral® (acebutolol), Zebeta® (bisoprolol), Brevibloc® (esmolol), Inderal® (propanolol), Tenormin® (atenolol), Normodyne®, Trandate® (labetalol), Coreg® (carvedilol), Lopressor®, or Toprol-XL® (metoprolol).

The ACE inhibitor of the combined pharmaceutical composition may be one or more of Lotensin® (benazepril), Capoten® (captopril), Vasotec® (enalapril), Monopril® (fosinopril), imidapril, Prinivil® (lisinopril), Zestril®, Univasc® (moexipril), Accupril® (quinapril), Aceon® (perindopril erbumine), Altace® (ramipril), or Mavik® (trandolapril).

The ARB of the combined pharmaceutical composition may be one or more of Atacand® (Candesartan cilexetil), Teveten® (Eprosartan), Avapro® (Irbesartan), Cozaar® (Losartan) Benicar® (Olmesartan medoxomil), Micardis® (Telmisartan) or Diovan® (Valsartan).

A factor of adrenal GRK2 inhibition as a HF therapy relates to tissue specificity. If a genetic approach is undertaken, as provided herein, this specificity may be achieved by delivering the GRK2 inhibitory transgene (e.g., βARKct peptide, GRK2 siRNA, GRK2 antisense oligonucleotide, etc.) through the suprarenal arteries or direct intra-suprarenal injection. Further concerning a pharmaceutical GRK2 inhibitor, extra-adrenal inhibition of GRK2 can be rather inevitable. Inhibition of cardiac GRK2, however, has been proven to increase contractility, so this could be beneficial in HF. Moreover, if administered concomitantly with beta-blockers, it would have minimal effect because the cardiac beta adrenergic system would be blocked anyway.

The use of an therapeutically effective amount of a GRK2 inhibitor disclosed herein for the treatment of HF, endocrinological, or non-endocrinological disease may comprise a pharmaceutical composition that may be in lactated Ringer's solution and the composition is sterile. Lactated Ringer's solution is a solution that is isotonic with blood and intended for intravenous administration. Included are antioxidants, buffers, antibiotics and solutes that render the compositions substantially isotonic with the blood of an intended recipient. In another embodiment, the composition comprises gene delivery vectors described herein. In another embodiment, the compositions also include water, polyols, glycerine and vegetable oils, and nutrients for cells, for example. Compositions adapted for parenteral administration can be presented in unit-dose or multi-dose containers, in a pharmaceutically acceptable dosage form. Such dosage forms, along with methods for their preparation, are known in the pharmaceutical and cosmetic art. See, e.g., REMINGTON'S PHARMACEUTICAL SCIENCES (Mack Pub. Co., 18th ed. 1990).

In one embodiment, dosage forms include pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol.

In one embodiment, other ingredients can be added, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

The following examples illustrate various methods for compositions in the treatment method of the invention. The examples are intended to illustrate, but in no way limit, the scope of the invention.

### EXAMPLES

### Example 1

Experimental Procedures: Adrenal chromaffin cell isolation and culture: The animal models of heart failure: mice with myocardial-targeted overexpression of calsequestrin (CSQ/Tg mice); male Sprague-Dawley rats (450-500 g; Harlan) ten weeks post-cryo-myocardial infarction (cryo-MI) (HF rats); as well as CSQ/βARKct mice, were previously established and characterized. Harding et al., 98 P.N.A.S. USA, 5809-14 (2001); Most et al., 114 J. Clin. Invest. 1550-63 (2004). Nontransgenic littermate mice were used as healthy controls for the mouse heart failure model. Chromaffin cells were isolated from adrenal glands excised from these animals and cultured as described previously in Brede et al., 2003. The purity of cultured chromaffin cells was assessed morphologically, and by immunofluorescence/immunoblotting for endogenous expression of tyrosine hydroxylase, and was over 90% in all experiments. Cells from separate isolations were used in each experiment.

In vivo adrenal gene delivery: All animal procedures and experiments were performed in accordance with the guidelines of the Institutional Animal Care and Use Committee of Thomas Jefferson University. For adrenal-specific in vivo gene delivery in HF rats, adenovirus was delivered directly to the adrenal gland using a surgical technique. Rats with chronic heart failure ten weeks after myocardial infarction were directly injected with virus into both of their adrenal glands, immediately after a transversal cut of the median abdomen was made and ventral visceral organs temporarily placed on the side so as to expose the retroperitoneal region. The procedure has minimal mortality and morbidity risks and results in specific gene delivery to the adrenal gland. HF rats were treated with either AdβARKct or AdGFP as a control. A separate group of heart failure rats were treated with adrenal injections of saline to control for adenovirus delivery; results in these rats were the same as in AdGFP-treated rats. For moxonidine treatment of HF rats, systemic administration of 1.0 mg/kg/d of moxonidine (delivered via a daily intraperitoneal injection) was initiated at the time of in vivo adrenal gene delivery and continued for seven days, at which time plasma was taken for determination of circulating catecholamine levels (see below). A dose of 0.5 mg/kg/d was also administered in separate cohorts of mice and rats; results were similar to those shown in FIG. 5F.

Echocardiographic and hemodynamic measurements: Two-dimensional guided M-mode and Doppler echocardiography using a 14-MHz transducer (Acuson Sequoia C256, Siemens Medical, Malvern, Pa.), and closed chest cardiac catheterization were performed in animals as described previously in Most et al., 2004. Three independent echocardiographic measurements were taken in both modes. Hemodynamic analysis included heart rate, maximal LV systolic pressure, end-diastolic pressure, and maximal and minimal first derivatives of LV pressure.

Adenoviruses: AdβARKct or AdGFP recombinant adenoviruses adenoviruses were purified as described previously in Most et al., 2004 using two sequential rounds of CsCl density gradient ultracentrifugation.

Plasma and in vitro catecholamine secretion measurements: Plasma Epi and NE levels were determined by ELISA, performed on rat and mouse plasma samples with the BI-CAT EIA kit from ALPCO Diagnostics (Windham, N.H.). In vitro Epi and NE secretion in the supernatant of cultured chromaffin cells was measured by use of the same ELISA kit essentially as described previously by Wilson & Kirschner, 3J. Neurosci. 1971-78 (1983). In vitro catecholamine (CA) secretion measurements. Briefly, 48 hrs post-isolation, chromaffin cells were placed in a balanced salts buffer and stimulated with 20 μM nicotine for 30 min, following pre-treatment with 10 μM UK14304 (Sigma-Aldrich) or vehicle for 30 min. At the end of nicotine treatment, the supernatant was collected for determination of its CA content. Adenoviral-transfected cells were infected 48 hrs post-isolation and cells were subjected to the various treatments 24 hrs after transfection (72 hrs post-isolation). In all experiments, the supernatants used for CA measurements were collected from cultures of equal numbers of cells as determined by cell counting and post-experiment protein determination in harvested cells.

RNA isolation, reverse transcription, and quantitative RT-PCR: Total RNA isolation, reverse transcription, and quantitative RT-PCR were carried out by the use of a MyIQ Single-Color Real-Time PCR detection system (BioRad, Hercules Calif.) and SYBR Green Supermix (BioRad) and 100 nM of gene-specific oligonucleotides as described in Pleger et al., 12 Mol. Therapy. 1120-29 (2005). All gene-specific oligonucleotide primer pairs used and their experimentally determined annealing temperatures are listed in Table 1.

TABLE 1
Primer sets for quantitative RT-PCR.

| | | |
|---|---|---|
| | | Anneal. |
| Gene | Primer pair used | Temp. |

| | | | |
|---|---|---|---|
| 18 S rRNA | Forward: 5′-TCAAGAACGAAAGTCGGAGG-3′ | (SEQ. ID. No. 1) | 60° C. |
| (mouse, rat) | Reverse: 5′-GGACATCTAAGGGCATCAC-3′ | (SEQ. ID. No. 2) | |
| GRK2 | Forward: 5′-CCCTCTCACCATCTCTGAGC-3′ | (SEQ. ID. No. 3) | 63° C. |
| (mouse, rat) | Reverse: 5′-CGGTTGGGGAACAAGTAGAA-3′ | (SEQ. ID. No. 4) | |
| GRK3 | Forward: 5′-GATATCGGCTCCTTCGATGA-3′ | (SEQ. ID. No. 5) | 63° C. |
| (mouse, rat) | Reverse: 5′-CAGTCCTTCCCCATAGCGTA-3′ | (SEQ. ID. No. 6) | |
| GRK5 | Forward: 5′-GAAGGTTAAGCGGGAAGAGG-3′ | (SEQ. ID. No. 7) | 63° C. |
| (mouse, rat) | Reverse: 5′-TCCAGGCGCTTAAAGTTCAT-3′ | (SEQ. ID. No. 8) | |
| Tyrosine | Forward: 5′-GAAGGGCCTCTATGCTACCCA-3′ | (SEQ. ID. No. 9) | 63° C. |
| Hydroxylase | Reverse: 5′-TGGGCGCTGGATACGAGA-3′ | (SEQ. ID. No. 10) | |
| (mouse, rat) | | | |
| α2A-AR | Forward: 5′-CATCAGCCTTGACCGCTACTG-3′ | (SEQ. ID. No. 11) | 58° C. |
| (mouse, rat) | Reverse: 5′-CGCACGTAGACCAGGATCATG-3′ | (SEQ. ID. No. 12) | |
| α2A-AR | Forward: 5′-GGCAAGACTTGGTGCGA-3′ | (SEQ. ID. No. 13) | 58° C. |
| (human) | Reverse: 5′-CGACGAGATGACGTACCACT-3′ | (SEQ. ID. No. 14) | |
| α2B-AR | Forward: 5′-TCATCCTCTTCACCATTTTCG-3′ | (SEQ. ID. No. 15) | 58° C. |
| (mouse) | Reverse: 5′-CTGGAAGCCAAGATGTACCAG-3′ | (SEQ. ID. No. 16) | |
| α2B-AR | Forward: 5′-CTCATCATCCCTTTCTCTCTG-3′ | (SEQ. ID. No. 17) | 58° C. |
| (rat) | Reverse: 5′-CCTCTTCATCTCCCTCCTCTG-3′ | (SEQ. ID. No. 18) | |
| α2B-AR | Forward: 5′-CCTGCGCATCTACCTGATCG-3′ | (SEQ. ID. No. 19) | 58° C. |
| (human) | Reverse: 5′-AAGCTGAGGCCGGAGACACT-3′ | (SEQ. ID. No. 20) | |
| α2C-AR | Forward: 5′-TTCACCGTGGTAGGCAAT-3′ | (SEQ. ID. No. 21) | 58° C. |
| (mouse) | Reverse: 5′-TTTCTCGCTGAGCGTACG-3′ | (SEQ. ID. No. 22) | |
| α2C-AR | Forward: 5′-CCTTCCCGCCTCTCGTCTCTT-3′ | (SEQ. ID. No. 23) | 58° C. |
| (rat) | Reverse: 5′-TCTCGCCGCCGTCCTCCTCTG-3′ | (SEQ. ID. No. 24) | |
| α2C-AR | Forward: 5′-GTGGTGATCGCCGTGCTGAC-3′ | (SEQ. ID. No. 25) | 58° C. |
| (human) | Reverse: 5′-CGTTTTCGGTAGTCGGGGAC-3′ | (SEQ. ID. No. 26) | |
| Spinophilin | Forward: 5′-AGCCTTGGAGAATGGCAGCAC-3′ | (SEQ. ID. No. 27) | 60° C. |
| (mouse) | Reverse: 5′-TTCATCGTCCTCCTCCAGGGC-3′ | (SEQ. ID. No. 28) | |
| Spinophilin | Forward: 5′-AGCCTTGGAGAATGGCAGCAC-3′ | (SEQ. ID. No. 29) | 60° C. |
| (rat) | Reverse: 5′-TTCATCGTCCTCTTCCAGGGC-3′ | (SEQ. ID. No. 30) | |
| β-arrestin 1 | Forward: 5′-AGCATGAGGACACGAATCTGGCT-3′ | (SEQ. ID. No. 31) | 60° C. |
| (mouse) | Reverse: 5′-CCGTCATCCTCTTCGTCCTTGTC-3′ | (SEQ. ID. No. 32) | |
| β-arrestin 1 | Forward: 5′-GTCAAAGTGAAGGTGGTGGTGTC-3′ | (SEQ. ID. No. 33) | 60° C. |
| (rat) | Reverse: 5′-CCATCATCCTCTTCGTCCTTGTC-3′ | (SEQ. ID. No. 34) | |
| β-arrestin 2 | Forward: 5′-GTCAAGGTGAAGCTGGTGGTGTC-3′ | (SEQ. ID. No. 35) | 60° C. |
| (mouse) | Reverse: 5′-ATAACCCAAGTAAGGTACCCTCT-3′ | (SEQ. ID. No. 36) | |
| β-arrestin 2 | Forward: 5′-TACAGGGTCAAGGTGAAGCTGGT-3′ | (SEQ. ID. No. 37) | 60° C. |
| (rat) | Reverse: 5′-GGTCATCACAGTCGTCATCCTTC-3′ | (SEQ. ID. No. 38) | |

RT-PCRs on mRNA isolated from mouse or rat brain, as a positive control for the expression of all three subtypes are also shown. No signals were observed in the absence of reverse transcriptase. Quantification of mRNA included normalization to 18s rRNA levels. Specific PCR products were determined using melting curves and gel electrophoresis as described Wilson & Kirschner, 3 J. Neurosci. 1971-78 (1983). Quantitative data are complied and shown as histograms.

Western blots to assess protein levels of mouse, rat and human tyrosine hydroxylase (AB152; Chemicon, Temecula, Calif.), GRK2 (sc-562; Santa Cruz Biotechnology), α-actin (A2066; Sigma-Aldrich, St. Louis, Mo.) and mouse and rat spinophilin (sc-14774; Santa Cruz Biotechnology) were done using protein extracts from excised and homogenized adrenal glands as described previously for myocardial protein extracts (Most et al., 2004; Iaccarino et al., 1998; Pleger et al., 2005). Specifically for GRK2, due to low endogenous expression and scarcity of adrenal tissue, immunoprecipitation of GRK2 was performed prior to western blotting of GRK2. Visualization of western blot signals were performed with Alexa Fluor 680—(Molecular Probes) or IRDye 800CW—coupled (Rockland Immunochem., Inc., Gilbertsville, Pa.) secondary antibodies on a LI-COR infrared imager (Odyssey, Li-Cor Bioscienses, Lincoln, Nebr.) and pictures were processed by Odyssey version 1.2 infrared imaging software. The western blots were quantified by densitometry and the data was presented as histograms. All densitometry scans were carried out in the linear range of detection, as described by Pleger et al., 12 Molecular Therapy 1120-29 (2005).

Saturation ligand binding: Plasma membranes from excised adrenal glands or hearts were prepared as described previously (Harding et al., 2001), and saturation binding was performed as described previously using α₂AR radioligands (Uhlen et al., 343 Eur. J. Pharmacol. 93-101 (1998)) or ¹²⁵I-CYP (iodocyanopindolol) for cardiac βARs. Harding et al., 2001. For mouse adrenals, the α₂AR-specific antagonist [³H] rauwolscine, relatively specific for the mouse α_{2C} subtype37 was used. For rat adrenals, another α₂AR-specific antagonist, [³H] RX821002, relatively specific for the rat α₂A subtype37 was used. Both α₂AR radioligands were from PerkinElmer (Wellesley, Mass.), whereas I-CYP was from Amersham Biosciences (GE Healthcare, Piscataway, N.J.). Data were analyzed by non-linear regression analysis using GraphPad Prism (GraphPad Software, Inc., San Diego, Calif.). Each independent experiment was performed with six adrenals pooled from three mice/litter or with four adrenals pooled from two rats/group each.

Immunofluorescence and immunohistochemistry: Immunofluorescence imaging of adrenal cross-sections was carried out as described previously (Most et al., 2004, supra). For co-immunofluorescence studies, rat or human adrenal cross-sections were fixed, permeabilized and labeled with monoclonal anti-tyrosine hydroxylase (MAB318; Chemicon) and rabbit polyclonal anti-GRK2 (sc-562; Santa Cruz Biotechnology) antibodies, followed by the corresponding Alexa Fluor 594 anti-mouse and Alexa Fluor 568 anti-rabbit secondary antibodies (Molecular Probes). Confocal images were obtained using a ×40 objective on a Leica Microsystems TCS SP laser scanning confocal microscope.

Calibration and assessment of intracellular Ca²⁺ transients in chromaffin cells in response to K⁺-induced depolarization were performed essentially as described. Brede, 2003; Zhou & Neher, 469 J. Physiol. 245-73 (1993). Changes in intracellular Ca²⁺ were monitored fluorimetrically using the Ca²⁺-sensitive probe Fura 2 (1 μM). The recording system included a Zeiss, Axiovert S170 inverted microscope fitted with epifluorescence (monochromator TILL Polychrome II, Germany). Cells were alternatively illuminated at 340 nm and 380 nm at a frequency of 140 Hz. Emission for each excitation wavelength was filtered at 510 nm and digitized using interface (EPC9, HEKA, Germany). The ratio 340/380 was displayed on-line using the Xchart software. Chromaffin cells isolated from HF or sham-operated rats were exposed to 10 μM UK14304 or vehicle for 30 min in culture medium containing a fixed Ca²⁺ concentration of 2.5 mM, and then 150 mM KCl was added to induce depolarization. Only round-shaped, healthy, and attached chromaffin cells with a resting 340/380 ratio lower than 1 were selected for measurement.

β-arrestin membrane recruitment: βarr1 recruitment to rat primary chromaffin cell membranes or whole adrenal gland-derived plasma membranes was measured by western blotting in membrane preparations, essentially as described previously in Perry et al., 198 Science 834-36 (2002). Cultured chromaffin cells, isolated from rat adrenal glands, were transfected with the indicated recombinant adenoviruses and 24 hrs post-infection they were treated with vehicle or 10 μM UK14304 for 8 min. The cells were then harvested and plasma membranes prepared by hypotonic lysis with 5 mM TrisCl, 5 mM EDTA, pH 7.4, and subsequent ultracentrifugation at 47.000×g. The membranes were resuspended in RIPA buffer, containing Triton 0.5% and protease inhibitors, and then fractions of membrane lysates containing 50 μg of protein were loaded for SDS-PAGE, and subsequently western blotted with an antibody against rat βarr1 and −2 (A1CT40, a generous gift from Dr. R. J. Lefkowitz, Duke Univ. Medical Center, Durham, N.C.). Plasma membrane preparations from whole adrenal glands excised from in vivo infected rats were obtained by the same procedure. Blotting for actin in the same membrane lysates was also performed as loading control. βarr1 membrane recruitment was quantified as the densitometric ratio of βarr1 and actin bands detected in each sample.

Statistical analysis: Data are generally expressed as mean±SEM. Unpaired 2-tailed Student's t test and one-way ANOVA were performed for statistical comparisons. For all tests, a p value of less than 0.05 was considered to be significant.

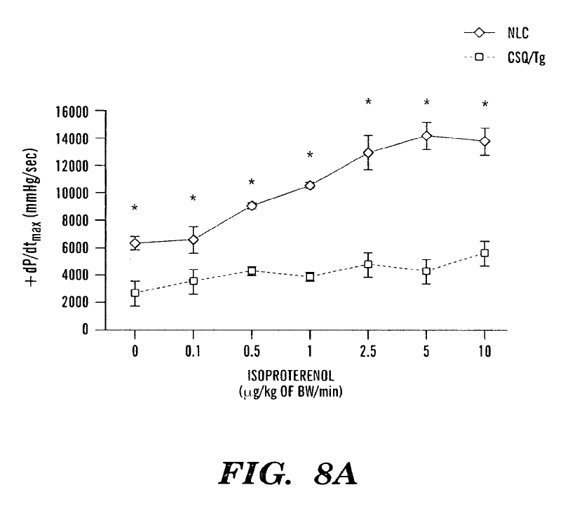

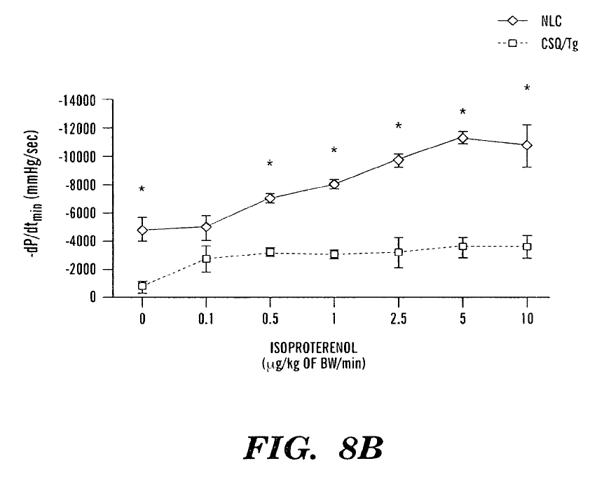

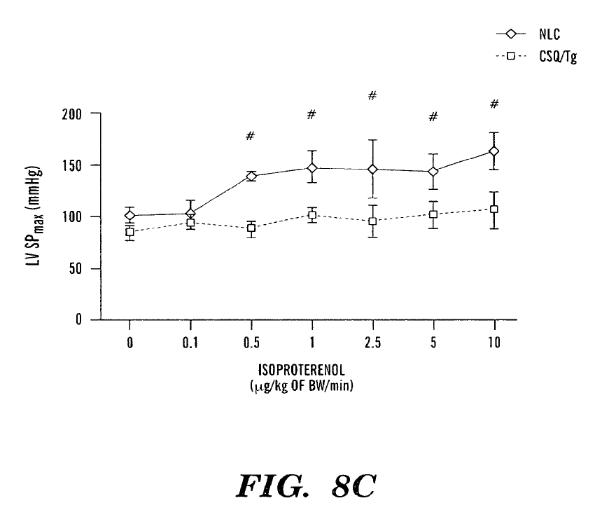

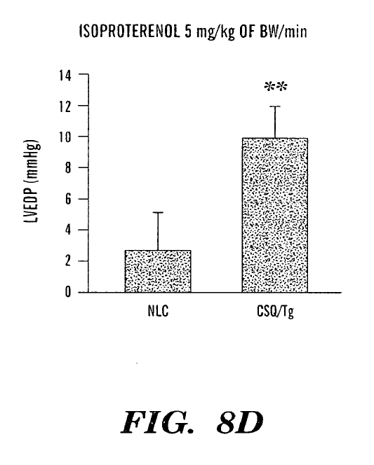

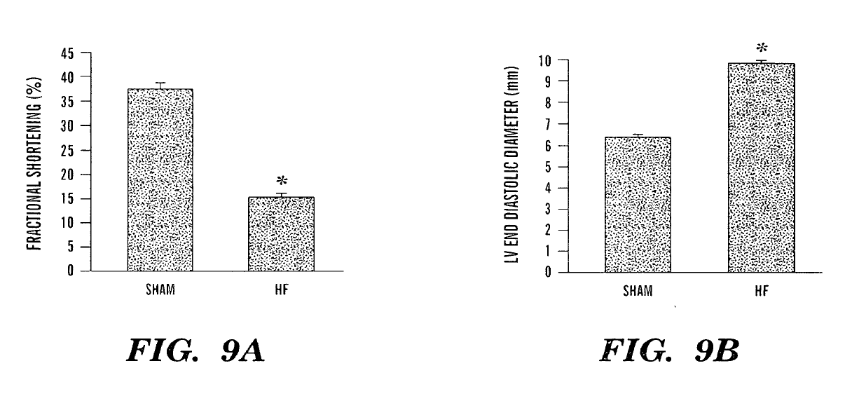

Adrenal hypertrophy and overactivity in HF: Two models of heart failure were used to study adrenal signaling (hypertrophy and over-activity): Transgenic (Tg) mice with cardiac-targeted over-expression of the sarcoplasmic reticulum calcium-binding protein calsequestrin (CSQ) (Harding et al., 9 P.N.A.S. USA 5809-14 (2001)) (CSQ/Tg mice or HF mice), and rats with chronic HF following myocardial infarction (MI) (HF rats). Most et al., 114 J. Clin. Invest. 1550-63 (2004). Three-month-old CSQ/Tg mice and ten week post-MI (HF) rats have significantly diminished cardiac function as assessed by both echocardiography and left ventricular (LV) catheterization. An increased in the left ventricular (LV) end-diastolic dimension (EDD) indicated dilatation and remodeling in CSQ/Tg mice and HF rats (FIG. 7A and FIG. 9B). A decrease in the cardiac ejection fraction indicated contractile failure in CSQ/Tg mice and HF rats (FIG. 7B and FIG. 9A). FIG. 8 shows that there is a loss of inotropic reserve and function in the CSQ/Tg mice used to study adrenal function.

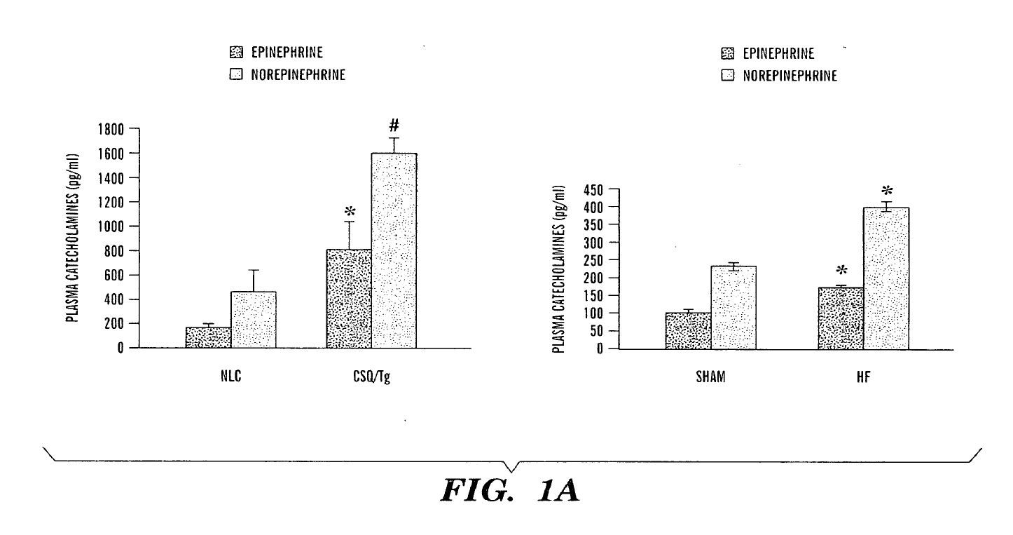

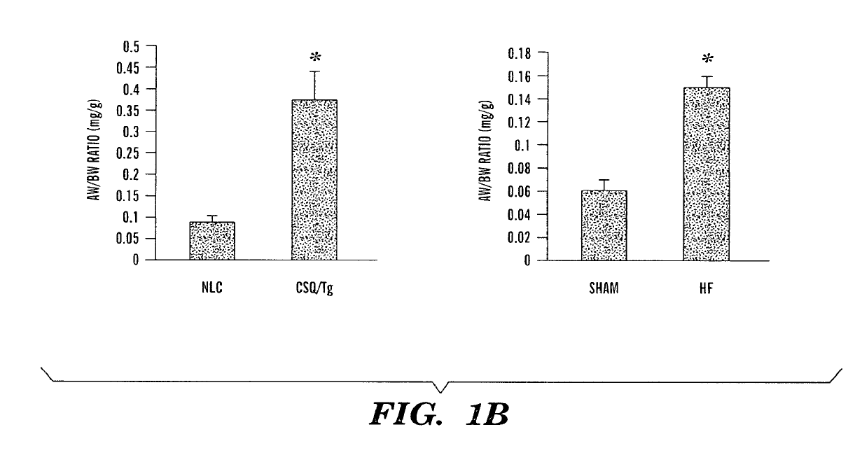

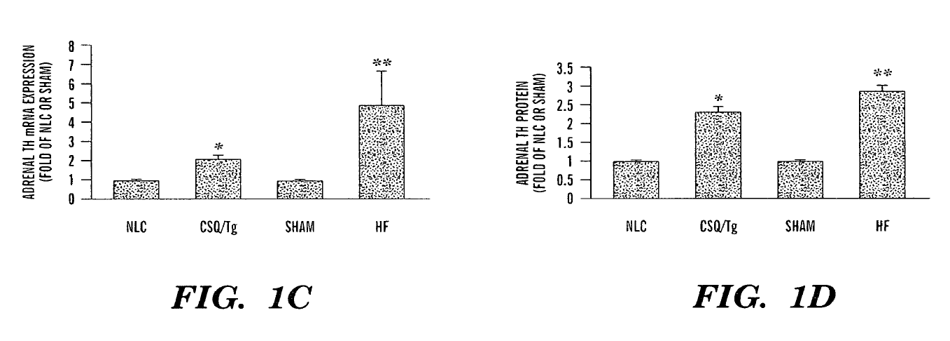

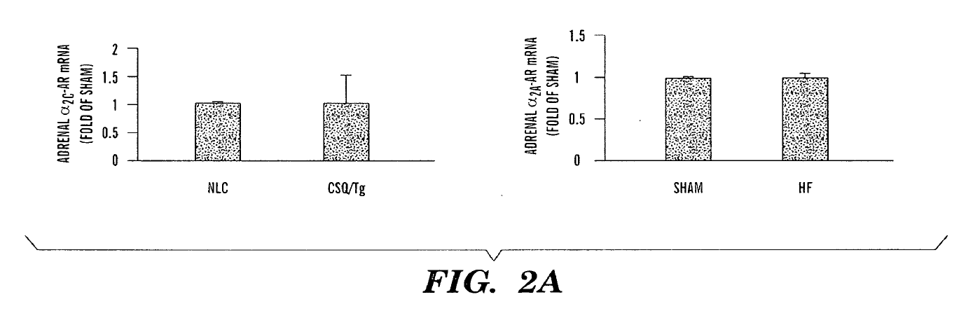

In both heart failure models, there were significantly elevated plasma levels of NEpi and Epi, indicating increased SNS outflow (FIG. 1A). Because the adrenal medulla is a key SNS component for overall catecholamine outflow, the functional status of the adrenal was examined. First, CSQ-Tg mice and heart failure rats both exhibited significant adrenal hypertrophy compared to age-matched healthy controls (FIG. 1B). In addition, the expression of tyrosine hydroxylase, the enzyme that catalyzes the rate-limiting step of catecholamine biosynthesis in adrenal chromaffin cells (Hoffman & Taylor, 2001; Young & Landsberg, 1998) was significantly elevated both at the mRNA (FIG. 1C) and protein (FIG. 1D) levels in HF mice and rats when compared to age-matched healthy controls. Each independent experiment performed consisted of six adrenal glands pooled from three mice/litter or four adrenal glands pooled from two rats/group each.

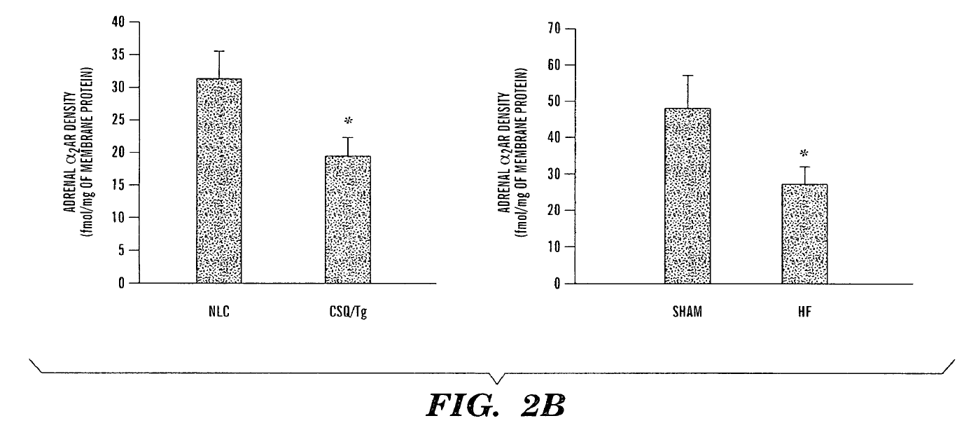

An aspect of the present invention addresses adrenal α₂AR down-regulation in HF. Because α₂ARs are the only type of adrenergic receptor responsible for inhibiting catecholamine secretion from the adrenal gland (Brede et al., Mol. Endocrinol. 17:1640-46 (2003)), the enhanced catecholamine levels in HF mice and rats could be due to diminished α₂AR function in the adrenal medulla and was thus investigated. First, using quantitative real-time RT-PCR, the levels of mRNA expression of α₂AR subtypes in adrenal glands isolated from CSQ-Tg mice and post-myocardial infarction rats were measured. In mouse adrenal glands, only mRNA for the α_{2C}AR subtype was detected, consistent with previous findings (Brede et al., 2003), and there was no difference in mRNA levels of this subtype in the adrenal glands of control (NLC) and CSQ-Tg mice (FIG. 2A). In rat adrenal glands, similar mRNA expression was found for only one of the three α₂AR subtypes, in this case α_{2A}AR (FIG. 2A). As in mice, no changes in mRNA levels of this subtype were found in adrenal glands of heart failure rats compared to sham controls (FIG. 2A). Notably, mRNA for only the α_{2A}AR subtype in human adrenal glands was detected. This indicates that the corresponding adrenal α₂AR subtype in humans is the α_{2A} subtype, which has been found to be a GRK2 substrate in vitro. Jewell-Motz & Liggett, 271 J. Biol. Chem., 18082-87 (1996); Kurose & Lefkowitz, 269 J. Biol. Chem. 10093-99 (1994).

Next, α₂AR density in plasma membranes isolated from adrenal glands in these heart failure models was assessed using saturation ligand binding studies with [³H]-labeled rauwolscine on plasma membrane preparations of adrenal glands excised from HF mice or with [³H]-labeled RX821002 on plasma membrane preparations of adrenal glands excised from HF rats. Non-specific binding was determined in the presence of 0.4 mM phentolamine. In CSQ/Tg mouse adrenal membranes, and in contrast to mRNA results, saturation ligand binding studies revealed significantly lower α₂AR density compared to NLC membranes (FIG. 2B). In HF rats, a similar ˜40% loss of receptor number compared to healthy control rats was found (FIG. 2B). Thus, there is significant, post-transcriptional α₂AR down-regulation in HF, and this appears to be independent of HF etiology and α₂AR subtype.

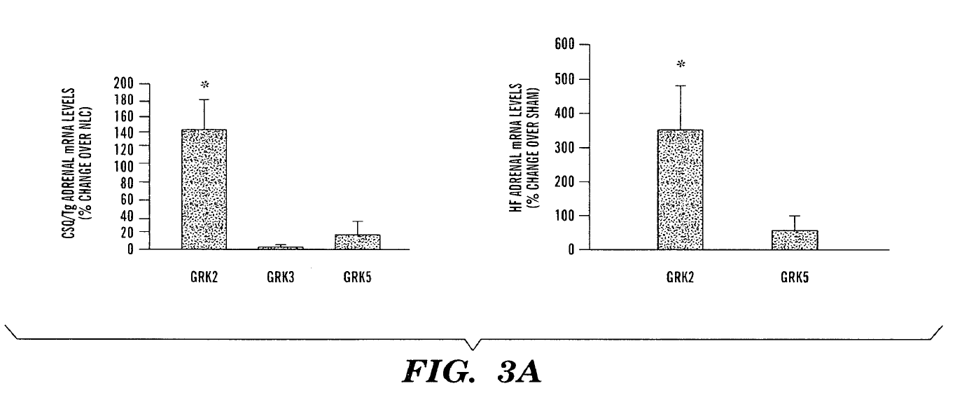

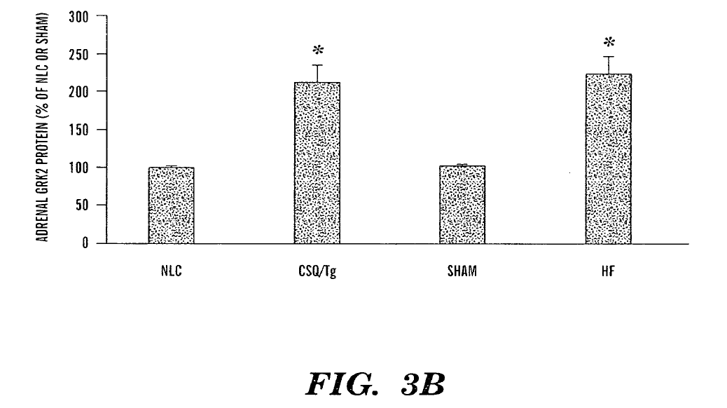

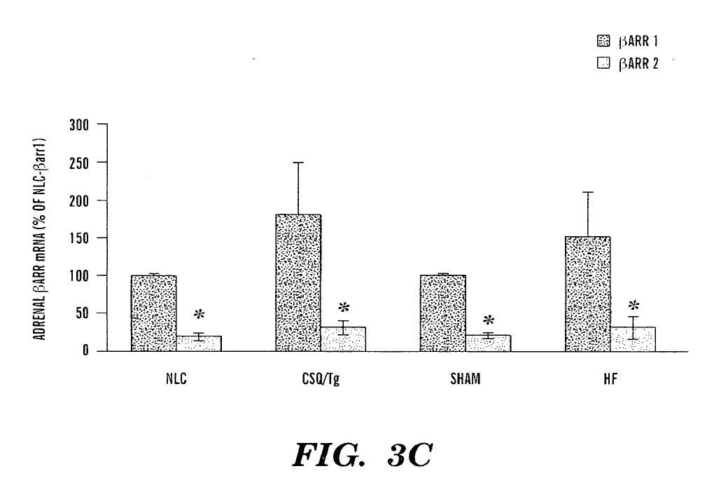

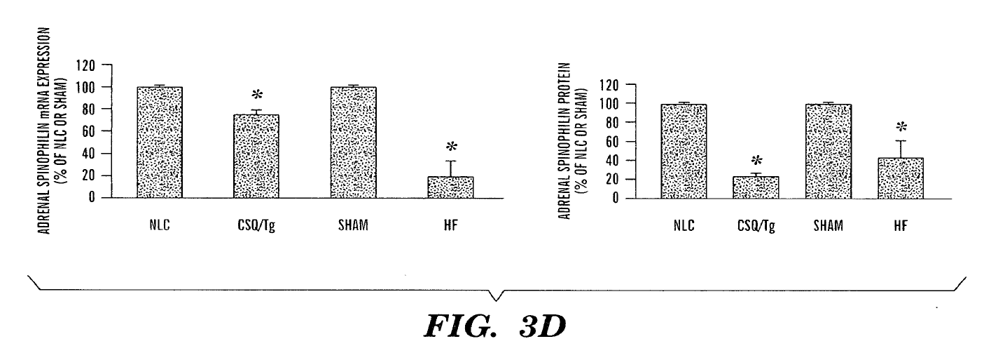

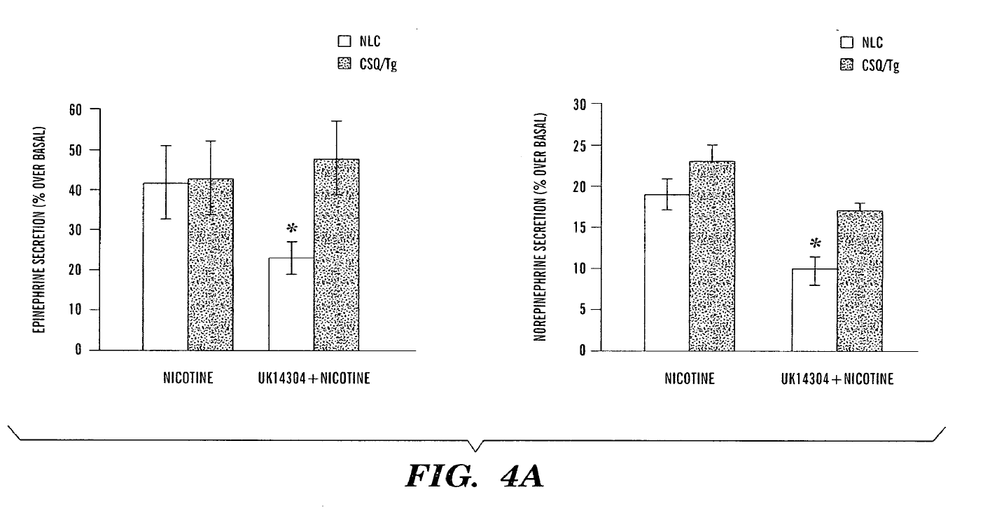

Another aspect of the present invention addresses adrenal GRK2 up-regulation in HF. More specifically, α₂AR down-regulation can be linked to the actions of GRKs, which would be similar to what occurs with α₂ARs in failing myocardium. Rockman et al., 415 Nature 206-12 (2002). There are no known studies, however, specifically addressing which GRKs are present in the adrenal gland and if they do indeed regulate α₂AR signaling. Importantly, some studies have shown that α₂ARs are substrates for GRK2. Jewell-Motz & Liggett, 1996; Kurose & Lefowitz, 1994. Accordingly, the expression of GRK2, GRK3 and GRK5 in the adrenal gland at the mRNA level via real-time PCR was addressed. The present disclosure reports for the first time that these three GRKs are endogenously expressed in the mouse adrenal gland, yet GRK3 was absent in rat adrenals (FIG. 3A). Interestingly, in the context of HF, there was a significant increase in mRNA expression for GRK2 in both the murine and rat models of cardiomyopathy (FIG. 3A). GRK3 and GRK5 were not altered in mouse HF and GRK5 was not changed in post-MI rat adrenals when compared to appropriate controls (FIG. 3A). Enhanced GRK2 expression was also seen at the protein level via protein immunoblotting of adrenal gland extracts in both mouse and rat HF models. Western blots for α-actin served as normalization control. Blots were densitometrically analysed (normalized to α-actin protein levels) and data reflected in histograms. *, p<0.05, compared to NLC or sham, n=three independent experiments performed with six adrenals pooled from three mice/litter or with four adrenals pooled from two rats/group each. Overall, these data indicate that adrenal GRK2 is significantly up-regulated in both models of HF, and the subsequent increase in GRK2 activity could be the trigger for the observed uncoupling and down-regulation of sympatho-inhibitory α₂ARs.

GRK2 protein was found in chromaffin cells of the adrenal medulla, based on co-immunofluorescence studies which showed co-localization with tyrosine hydroxylase. GRK2 presence in chromaffin cells was also confirmed by immunohistochemistry. Immunoblotting studies detected GRK2 in human adrenal gland, and co-immunofluorescence studies with tyrosine hydroxylase confirmed GRK2 localization in human adrenal medulla chromaffin cells. Consistent with the mRNA data, increased levels of GRK2 protein was found in adrenal gland extracts from both models of heart failure compared to controls (FIG. 3B). These data indicate that adrenal GRK2 is up-regulated in HF, potentially triggering the observed down-regulation of α₂ARs.

In addition to the actions of GRKs, α-arrestins are involved in directing desensitization and down-regulation of G-protein coupled receptors (GPCRs). Lefkowitz & Whalen, 16 Curr. Opin. Cell Biol. 162-68 (2004). β-arrestins bind to GRK-phosphorylated GPCRs and direct their internalization that leads to recycling (resensitization), down-regulation (via degradation) or to novel G-protein-independent signaling pathways. The latter may be due to the protein-scaffolding properties of β-arrestins. To complete the analysis of the status of adrenal α₂AR regulation, expression levels of β-arrestin1 (βarr1) and β-arrestin2 (βarr2) in the adrenal gland of control and HF animals were examined. Based on mRNA levels, βarr1 is the most abundant isoform in both species; however, heart failure did not alter the expression of either βarr (FIG. 3C). PCR gels were conducted with data obtained from five-six mice/litter or five-six rats/group and the histograms of the PCR gels after densitometry quantification are shown, including 18S rRNA levels as normalization control. No signals were observed in the absence of reverse transcriptase.

Of interest, α₂AR function and desensitization have been shown to be regulated by the protein spinophilin, which has recently been reported to regulate α₂AR function and signaling by antagonizing βarr actions at this receptor. Wang et al., 304 Science 1940-44 (2004). Spinophilin has never been examined in the adrenal gland. RT-PCR quantification and western blotting for adrenal spinophilin were performed. The 18S rRNA and α-actin were used as normalization controls, along with RT-PCR quantification normalized to 18S rRNA levels and densitometric analysis normalized to α-actin levels. The data represented n=7 mice or rats/group for PCR analysis, n=3 independent experiments performed with six adrenals pooled from three mice/litter or with four adrenals pooled from two rats/group each, for western blot analysis. There was a significant decrease in spinophilin mRNA expression compared to age-matched healthy control animals in both heart failure models (FIG. 3D). Western blotting confirmed a down-regulation of adrenal spinophilin protein in both HF mice and HF rats (FIG. 3D). The loss of this βarr antagonist at the α₂AR could also contribute to an overly uncoupled sympatho-inhibitory signaling of this receptor.

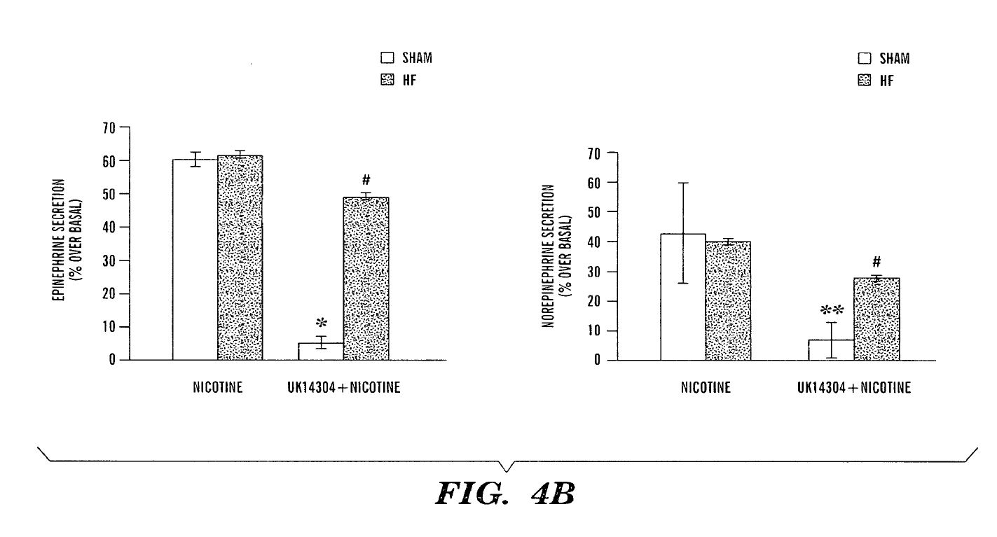

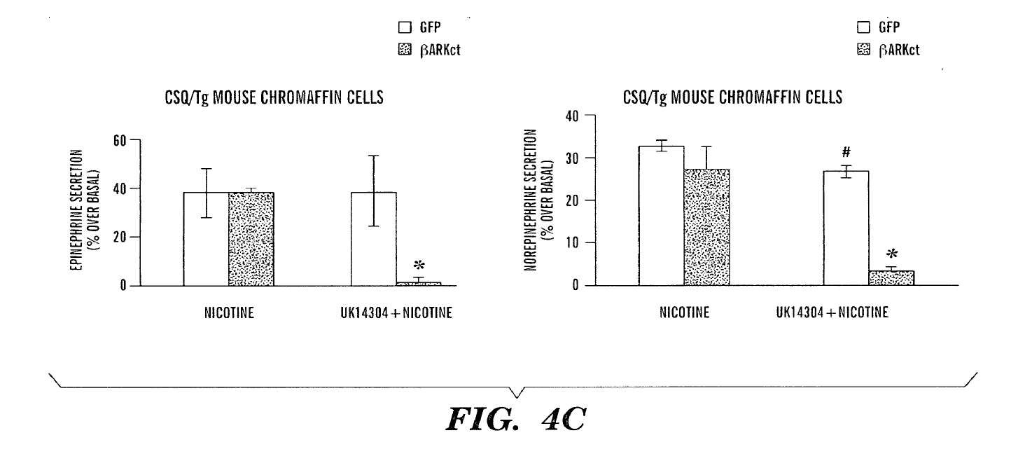

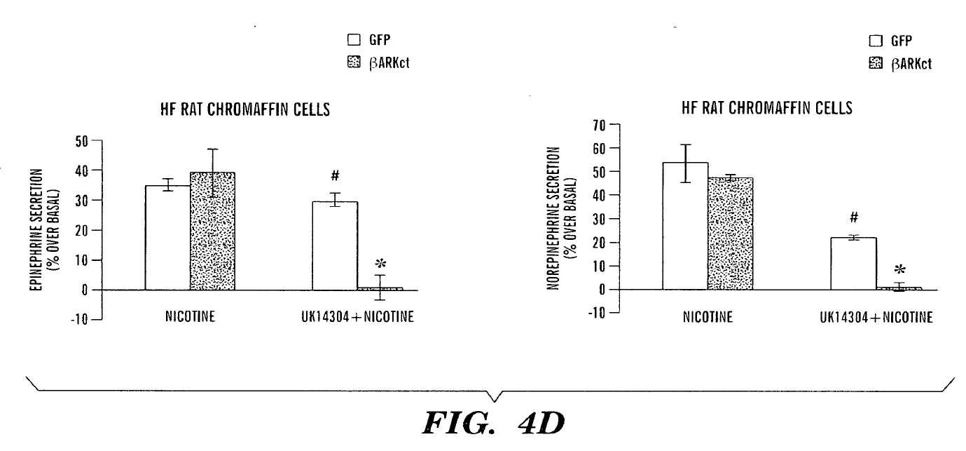

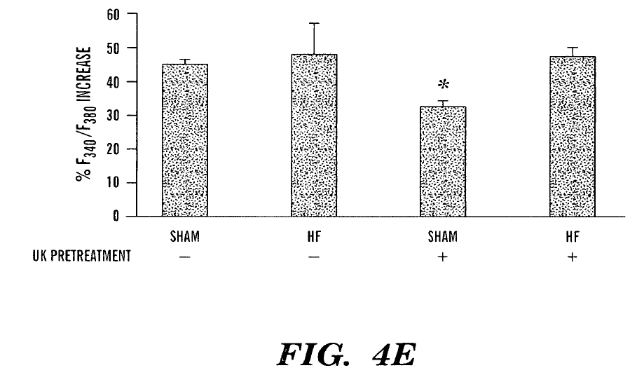

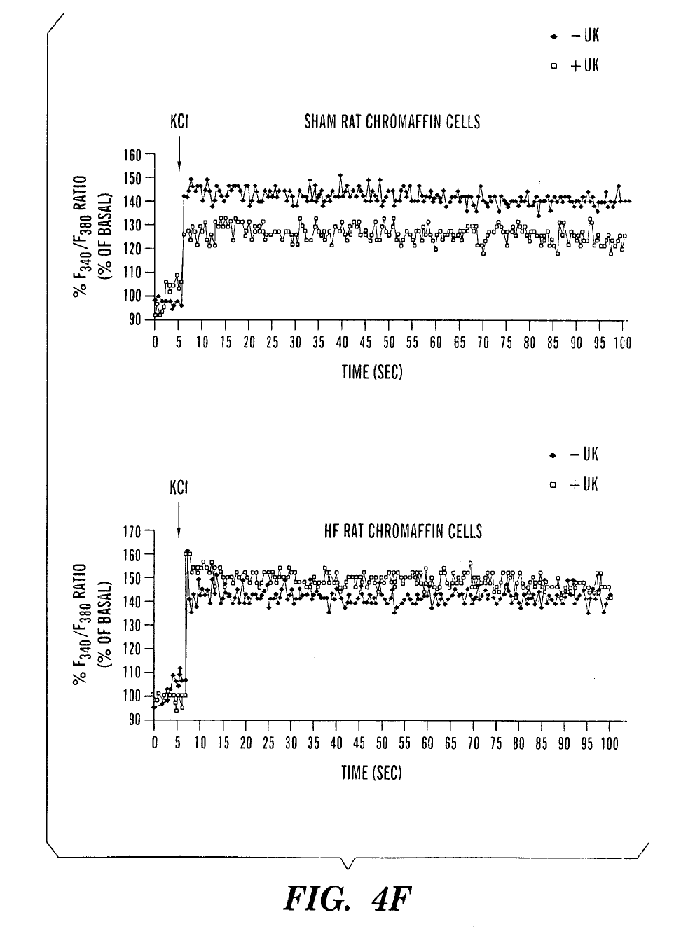

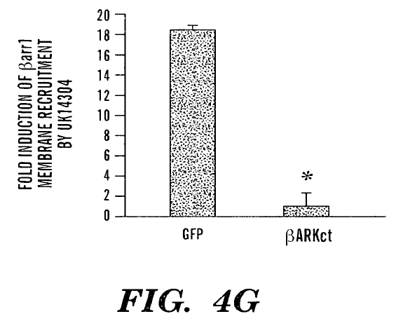

Yet another aspect of the present invention addresses the functional α₂AR consequences of GRK2 up-regulation in adrenal chromaffin cells in HF (GRK2-induced α₂AR dysfunction in failing chromaffin cells). To determine the functional consequences of the above adrenergic and α₂AR derangements in the adrenal gland in HF, the amount of catecholamine released from isolated and cultured chromaffin cells from the two mouse and rat HF models were compared with that from healthy, control animals. As shown in FIG. 4A, in CSQ/Tg mouse-derived chromaffin cells, the α₂AR-specific agonist UK14304 completely fails to inhibit both Epi and NE secretion in response to nicotine, while, in control cells, this α₂AR agonist attenuates catecholamine release from this nicotine-induced physiological stimulus. UK14304 pre-treatment alone had no effect. The data was from two independent experiments performed with triplicate samples, in cells derived from five mice (ten adrenals)/litter each.

Similarly, in HF rat chromaffin cells, UK14304 fails to inhibit secretion of both catecholamine compared to the inhibitory effects of α₂AR stimulation in sham-control chromaffin cells (FIG. 4B). Importantly, the use of nicotine in the absence of UK14304 pre-treatment led to similar secretion of Epi and NE in both heart failure and control cells (FIG. 4A and FIG. 4B). Thus, in addition to downregulation of α₂ARs in HF, the remaining membrane α₂ARs are functionally uncoupled, as they seem to be unable to inhibit catecholamine secretion. The data was from two independent experiments performed with triplicate samples, in cells derived from four rats (eight adrenals)/group each.

That the enhanced GRK2 activity is responsible for the loss of the inhibition of catecholamine secretion in HF chromaffin cells was shown using the well-characterized peptide inhibitor of GRK2 activation, the βARKct. Koch et al., 268 Science 1359-53 (1995). The βARKct inhibits GRK2 activity by competing for the membrane translocation/activation of this GRK directed by binding to the membrane-embedded βγ subunits of G-proteins. Adenoviral-mediated delivery of the βARKct to cultured chromaffin cells led to a complete restoration of UK14304-mediated inhibition of nicotine-induced Epi and NE secretion in both CSQ/Tg mouse (FIG. 4C) and HF rat (FIG. 4D) chromaffin cells, whereas control infection with a GFP adenovirus had no effect, as HF cells still had a loss of α₂AR-mediated CA secretion inhibition. UK14304 pre-treatment alone had no effect. The data was from two independent experiments performed with triplicate samples, in cells derived from five mice (ten adrenals)/condition each and from four rats (eight adrenals)/condition each.

Specifically, βARKct dramatically decreased the EC₅₀ of UK14304-mediated inhibition of catecholamine secretion both in CSQ/Tg mouse-derived chromaffin cells (Epi secretion: from 5.5±1.3 μM (mean±s.e.m.) in AdGFP cells to 200±1.5 nM in AdβARKct cells; NEpi secretion: from 8.0±2.63 μM in AdGFP cells to 50±1.4 nM in AdβARKct cells; P<0.05, n=6) and in HF rat chromaffin cells (Epi: from 6.4±2.5 μM in AdGFP cells to 100.0±5.2 nM in AdβARKct cells; NEpi: from 680.0±25.1 nM in AdGFP cells to 110±1.2 nM in AdβARKct cells; P<0.05, n=6). This strongly suggests that βARKct reverses α₂AR desensitization in these cells. βARKct expression in infected chromaffin cells was verified by confocal imaging and western blotting. These cells showed expression of endogenous tyrosine hydroxylase (TH), thus verifying the identity of these cells as being chromaffin cells, and for the βARKct peptide, verifying the expression of the peptide in AdβARKct-infected cells, but not in AdGFP-infected cells.

α₂ARs inhibit catecholamine secretion in chromaffin cells by inhibiting depolarization-evoked Ca²⁺ entry into the cells, which is absolutely necessary for subsequent exocytosis and catecholamine-containing vesicle fusion with the plasma membrane, the underlying process of catecholamine secretion. Brede, 2003; Zou & Neher, 469 J. Physiol., 245-73 (1993). Therefore, the cellular Ca²⁺ entry in HF rat and sham-control chromaffin cells was also examined. Pretreatment of control cells with UK14304 significantly inhibited depolarization-induced Ca²⁺ entry, as would be expected for maximal α₂AR activation in this process (FIG. 4E). A representative tracing of the Ca²⁺ transients is shown in FIG. 4F, and it is evident that in HF chromaffin cells, UK14304 fails to significantly alter Ca²⁺ entry (FIG. 4E and FIG. 4F), which is consistent with the catecholamine secretion studies above indicating that adrenal α₂ARs in HF are desensitized.

Because functional receptor desensitization is dependent on βarr binding to the receptor, βarr recruitment to the plasma membrane of primary cultured rat chromaffin cells, in response to stimulation with UK14304, was studied. Western blotting in membrane preparations from these cells infected with GFP showed a large βarr1 recruitment to chromaffin cell membranes in response to α₂AR stimulation, which was completely abrogated by βARKct expression (FIG. 4G). Taken together, these results demonstrate that GRK2 activity on adrenal α₂ARs induces βarr1 recruitment and binding, leading to desensitization which is enhanced in HF, resulting in dysregulation of catecholamine secretion and high catecholamines levels. In HF, enhanced GRK2-mediated desensitization results in α₂AR dysregulation and high plasma levels of NE and Epi. Western blots were quantified by densitometry and normalized to α-actin protein levels. N=three independent experiments, performed with cells isolated from six adrenals pooled together from three rats each.

Another aspect of the invention relates to the inhibition of adrenal GRK2 activity in vivo, which improves catecholaminergic responsiveness and function of the failing heart. Having shown that inhibition of GRK2 in chromaffin cells leads to restoration of adrenal α₂AR-mediated inhibition of catecholamine secretion, the in vivo inhibition of adrenal GRK2 was studied and its influence on SNS function and circulating circulating catecholamine levels and cardiac function in chronic HF were examined. The adrenal glands of rats having chronic HF ten weeks post-MI were infected in vivo with either with βARKct-encoding adenovirus (βARKct) or with control GFP-encoding adenovirus, and the cardiac function of these animals studied, followed by sacrifice and molecular analysis of their hearts and adrenals at seven days post-in vivo infection. Preliminary experiments in normal rats indicated that this day is the optimal post-infection time point for studying the effects of the adrenal adenoviral gene delivery, as assessed by a large transgene expression in the adrenal gland at seven days post-infection. HF rat adrenals were also treated with saline, as control for the in vivo gene delivery procedure. After sacrifice, a molecular analysis of adrenergic receptor signaling components in both the adrenal glands and myocardium was performed.

There was robust βARKct expression specifically in the adrenal glands of AdβARKct-treated rats (FIG. 5A); transgene expression was not detected in the heart, liver, kidney or lung tissue. In addition, adrenal-specific βARKct expression led to a significant decrease in total adrenal GRK2 and tyrosine hydroxylase levels compared to those in control AdGFP-treated rats (FIG. 5A), indicating that in vivo βARKct expression reverses the upregulation of adrenal GRK2 and tyrosine hydroxylase observed in HF. Further, there was decreased βarr1 membrane recruitment (FIG. 5A), consistent with decreased GRK2 activity at receptors. Notably, 7d after AdβARKct delivery to the adrenal gland, HF rats had a significant reduction in circulating levels of both NEpi (75±21% reduction, n=6, P<0.05) and Epi (51±10% reduction, n=6, P<0.05) compared to AdGFP-treated heart failure rats (also see below). Thus, adrenal GRK2 inhibition in vivo led to a significant reduction of SNS activity and outflow in HF. At 7d after adrenal AdβARKct delivery, there was evidence of molecular ‘reverse remodeling’ in the failing myocardium, with decreased GRK2 levels (FIG. 5B) and increased βAR density (FIG. 5C).

Lower GRK2 levels and increased βAR density indicate improved βAR signaling in the failing rat myocardium, probably due to lower catecholamine levels. Accordingly, significantly enhanced cardiac function and inotropic reserve were observed in heart failure rats after AdβARKct delivery to the adrenal gland (FIG. 5D, FIG. 5E). Compared to AdGFP-treated heart failure rats, adrenal βARKct expression led to significant in vivo improvements in ejection fraction 1 week after gene delivery (FIG. 5D). Further, adrenal AdβARKct delivery led to significantly improved basal and isoproterenol-stimulated left ventricular +dP/dtₘₐₓ and −dP/dtₘᵢₙ, measures of cardiac contractility and relaxation, respectively (FIG. 5E). Results in HF rats with saline-injected adrenal glands were similar to those in AdGFP-treated HF rats. Table 2 presents complete in vivo cardiac functional parameters in heart failure rats one week after AdβARKct delivery:

TABLE 2
In vivo cardiac functional parameters after adrenal gene delivery.

| | | |
|---|---|---|
| sham-operated | HF/AdGFP | HF/AdβARKct |
| (n = 8) | (n = 10) | (n = 10) |

Echocardiographic parameters after gene delivery

| | | | |
|---|---|---|---|
| LVIDs (mm) | 3.76 ± 0.08 | 8.33 ± 0.08^{#} | 7.35 ± 0.17^{#,}* |
| LVIDd (mm) | 6.36 ± 0.15 | 9.85 ± 0.06^{#} | 9.33 ± 0.16^{#,}* |
| FS (%) | 37.09 ± 1.39 | 15.35 ± 0.36^{#} | 18.53 ± 0.49^{#,}* |
| EF (%) | 65.01 ± 1.77 | 31.01 ± 0.74^{#} | 36.73 ± 0.84^{#,}* |
| PWTd (mm) | 1.25 ± 0.08 | 2.44 ± 0.1^{#} | 2.39 ± 0.1^{#,}* |

Basal LV Hemodynamic Measurements

| | | | |
|---|---|---|---|
| HR (min−1) | 318.2 ± 13.9 | 308.3 ± 11.5^{#} | 310.0 ± 11.7^{#} |
| LV + dP/dtₘₐₓ | 5,907 ± 446 | 4,145 ± 436^{#} | 4,535 ± 344^{#} |
| (mm Hg/s) | | | |
| LV − dP/dtₘᵢₙ | −5,598 ± 448 | −3,592 ± 217^{#} | −3626 ± 119^{#} |
| (mm Hg/s) | | | |
| LVESP | 120.6 ± 1.1 | 101.4 ± 1.5^{#} | 104.7 ± 1.6^{#} |
| LVEDP | 1.7 ± 0.6 | 12.2 ± 0.8^{#} | 10.7 ± 1.4^{#} |

Hemodynamic Measurements after maximal Isoproterenol
(333 ng/kg BW)

| | | | |
|---|---|---|---|
| HR (min−1) | 392.2 ± 13.2 | 380.2 ± 9.3 | 381.75 ± 10.0 |
| LV + dP/dtₘₐₓ | 12,528 ± 591 | 5,930 ± 448^{#} | 8,386 ± 403^{#,}* |
| LV − dP/dtₘᵢₙ | −6,981 ± 415 | −3,815 ± 280^{#} | −5221 ± 212^{#,}* |
| LVESP | 127.9 ± 1.8 | 109.2 ± 1.6^{#} | 118.7 ± 1.6^{#,}* |
| LVEDP | 2.5 ± 0.6 | 16.6 ± 0.8^{#} | 12.25 ± 0.5^{#,}* |

Phenotypic data:

| | | | |
|---|---|---|---|
| HW/BW (mg/g) | 2.03 ± 0.16 | 3.2 ± 0.12^{#} | 2.97 ± 0.06^{#} |
| AW/BW (mg/g) | 0.06 ± 0.01 | 0.15 ± 0.01^{#} | 0.14 ± 0.01^{#} |

Adrenal gene delivery of AdGFP (HF/AdGFP) or of AdβARKct (HF/AdβARKct) was performed at 10 weeks post-MI and the above parameters were measured 7 days post-gene delivery (direct adrenal medulla injection). Values of age-matched sham-operated animals are shown for comparisons.
LV + dP/dtmax, maximal first derivative of LV pressure rise;
LV − dP/dtmin, minimal first derivative of LV pressure fall;
HR, heart rate;
LVESP, LV end systolic pressure;
LVEDP, LV end diastolic pressure;
LVIDd, LV inner diameter during diastole;
LVIDs, LV inner diameter during systole;
FS, fractional shortening;
EF, ejection fraction;
PWTd, posterior wall thickness in diastole;
HW/BW: Heart weight-to-body weight ratio;
AW/BW: Adrenal weight-to-body weight ratio;
LV: Left ventricular;
^{#}p < 0.05 vs. sham, and
*p < 0.05 vs. HF/AdGFP. ANOVA with Bonferroni test were performed among all groups.
Data are presented as mean ± SEM.

These results indicate that adrenal GRK2 inhibition leads to a marked reduction of sympathetic stimulation of the failing heart, which in turn substantially improves cardiac adrenergic responsiveness and inotropic reserve and function to nearly normal levels.

The improvement of the in vivo sympatholytic effect of the α₂AR agonist moxonidine upon GRK2 inhibition was investigated. After 7d of moxonidine administration (1 mg/kg/d given intraperitoneally) in adrenal AdGFP-treated HF rats, circulating NEpi levels were lowered, but there was virtually no effect on Epi levels (FIG. 5F). In adrenal AdβARKct-treated HF rats, however there was a significant reduction in both NE and Epi plasma levels, which were further significantly reduced by moxonidine treatment (FIG. 5F). The effects of moxonidine in adrenal AdGFP-treated HF rats were similar to those in adrenal saline-treated HF rats.

The data provided herein presents the first molecular evidence that SNS activity of the adrenal gland is significantly enhanced in HF, and that this contributes significantly to the diminishment of cardiac inotropic reserve and function. This includes increased catecholamine biosynthesis as indicated by up-regulation of tyrosine hydroxylase and adrenal hypertrophy, which is consistent with hyper-activity. Enhanced SNS activity of the adrenal gland is especially important since catecholamine levels are a strong prognostic indicator of morbidity and mortality in HF. Cohn et al., 311 N. Engl. J. Med. 819-23 (1984). Perhaps more important than this SNS gain-of-function of the adrenal gland in HF is the novel finding that sympatho-inhibitory α₂AR function is significantly dysregulated, which appears to contribute significantly to enhanced catecholamine levels in HF.

The current findings indicate that the up-regulation of GRK2 in chromaffin cells of the adrenal medulla is the primary mechanism for the lack of α₂AR-mediated inhibition of catecholamine release because inhibition of this GRK in HF adrenal chromaffin cells results in restoration of normal regulation and inhibition of catecholamine release through α₂ARs. Overall, adrenal α₂AR function was deranged due to not only the enhanced GRK2-mediated desensitization, but the significant receptor down-regulation, increased membrane recruitment of βarr1, and decreased levels of the α₂AR-regulating protein, spinophilin. Importantly, it appears that this mechanism of adrenergic dysfunction is global: independent of the cause of HF, of the particular species, or of the specific adrenal α₂AR subtype involved, because it was found that this mechanism was at play in both rat and mouse HF models, which differ in terms of disease etiology, disease progression and severity, and individual adrenal α₂AR subtype expression. Interestingly, it was found that α₂ARs of the α_{2A} subtype are expressed along with GRK2 in the adrenal glands of humans as well. Given that human α_{2A}AR is a very well-known GRK2 substrate (Jewell-Motz & Liggett, 271 J. Biol. Chem. 18082-87 (1996); Kurose & Lefkowitz, 269 J. Biol. Chem. 10093-99 (1994)), it is possible that the herein reported mechanism of adrenal catecholamine hypersecretion in HF is at play also in human chronic HF.

### Example 2

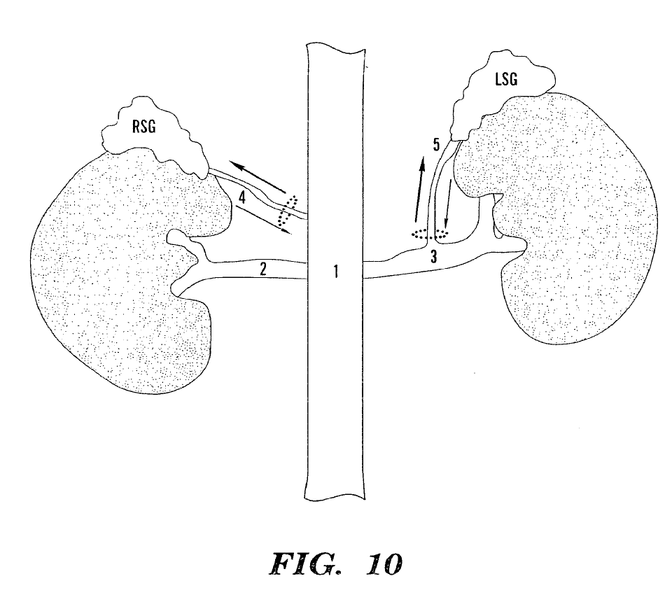

In vivo adrenal gene delivery: All animal procedures and experiments were performed in accordance with the guidelines of the IACUC of Thomas Jefferson University. For adrenal-specific in vivo gene delivery, we developed a novel surgical technique of retrograde delivery through the suprarenal vein. See FIG. 10. Note the difference between the venous circulations of the two adrenal glands in the rat. LSG: Left Suprarenal Gland, RSG: Right Suprarenal Gland, 1: Inferior vena cava, 2: Right renal vein, 3: Left renal vein, 4: Right suprarenal vein, 5: Left suprarenal vein. Thick arrows indicate direction of blood flow and thin arrows indicate direction of adenoviral injection (retrograde delivery). The black-dotted circles indicate the sites of the vein ligations preceding the virus injection. Rats undergo a transversal cut of the median abdomen, ventral visceral organs are temporarily placed on the side to expose retroperitoneal region. Subsequently, each suprarenal vein is permanently closed with a 5.0 thread at its respective origin and a P— 10 catheter is introduced directly into the suprarenal vein. The catheter tip is driven up to the gland and direct injection of the transgene follows (˜50 μl of adenovirus per each gland on average). After injection, the catheter is removed, visceral organs are hydrated with saline and placed back to their original positions, and the abdomen is closed. In addition to this intra-vascular technique, direct intra-adrenal delivery was also performed as previously example, which involves direct insertion of a 32G needle inside the gland before injection of the virus. Both techniques carry negligible mortality and morbidity risk and are extremely time-efficient (total duration ˜15 min per rat).

In vivo gene delivery efficiency: Efficiency of in vivo gene transfer was assessed in cryosectioned adrenal glands (15 μm) by expression of β-galactosidase (LacZ staining) as described previously (Pleger et al., 12 Mol. Ther. 1120-29 (2005)) and by GFP fluorescence (510 nm) using an Olympus IX81 confocal microscope. To confirm specificity of the GFP emission, the same offset for suppressed background excited at 488 nm was applied to all adrenals treated and additional measurements were taken below and above the GFP excitation spectrum. Additionally, the percentage of GFP-stained adrenal sections was assessed seven days following in vivo gene transfer within twenty-six randomly assigned visual fields using an Olympus IX 71 microscope, a mercury arc light, and suitable filters.

Western blots to assess protein levels of GRK2 and ARKct (sc-562; Santa Cruz Biotech.) and GAPDH (MAB374; Chemicon) were done using protein extracts from excised and homogenized adrenal glands and other organs as described. Lymperopoulos et al., 13 Nat. Med. 513-23, (2007); Pleger, et al., 2005; Harding et al., 2001). Visualization of western blot signals was performed with Alexa Fluor 680 (Molecular Probes) or IRDye 800CW-coupled (Rockland Inc.) secondary antibodies on a LI-COR infrared imager (Odyssey) and pictures were processed by Odyssey version 1.2 infrared imaging software.

As a means of targeted transgene expression in vivo to the adrenal gland, direct injection of recombinant adenovirus (Ad) vectors containing various transgenes into the suprarenal glands of normal three-month-old male Sprague-Dawley rats was performed. In addition, an alternative method based on retrograde delivery of adenoviruses through the suprarenal vein (FIG. 10) was developed. Because all the blood from both the cortex and the medulla of the adrenal gland drains into the suprarenal vein (Breslow 162L Am. J. Physiol. H1317-30 (1992)), this intra-vascular method of delivery could be an equally effective mode of gene transfer to the entire adrenal gland and one with potential for clinical application as a percutaneous method. Thus, both methods were tested for in vivo adrenal gene delivery.

The efficiency of these two different techniques was compared by quantitative staining/imaging of the marker genes β-galactosidase (β-gal) or GFP transgene expression after injection of the vectors AdLacZ or AdGFP, respectively. β-gal staining of AdLacZ-infected rat adrenal glands at 7d post-delivery showed that both techniques resulted in a robust and global transgene expression (90-95% of the whole adrenal gland on average, n=6) and importantly, gene expression can be seen throughout both the cortical and medullar regions. Visualization of GFP fluorescence 7d after AdGFP delivery confirmed the vast magnitude of transgene expression covering almost the entirety of the suprarenal gland obtained with both delivery techniques, similar to that seen after β-gal staining in AdLacZ treated adrenal glands.

Adrenal-restricted transgene expression by the two adrenal-targeted delivery methods. The tissue specificity of the adrenal-targeted in vivo gene delivery methods was examined to verify that gene delivery via these two methods does not lead to ectopic (extra-adrenal) transgene expression. Both AdβARKct as well as AdGFP were delivered. βARKct is not endogenously expressed in mammalian cells. At 7d post-adrenal AdβARKct delivery, the animals were sacrificed, their adrenal glands and various other tissues (hearts, lungs, livers, kidneys, spleens, etc.) were isolated, and performed western Blotting for βARKct expression. The βARKct peptide was robustly present in the adrenal glands via both methods, but, importantly, neither delivery method led to appreciable or detectable levels of the βARKct in any extra-adrenal tissues. In addition, no fluorescent signal could be detected in the extra-adrenal tissues (e.g. hearts, kidneys, liver) of AdGFP-infected rats. These data indicate that both the direct intra-adrenal and the intra-vascular suprarenal vein-mediated delivery techniques are highly specific for the adrenal gland tissue.

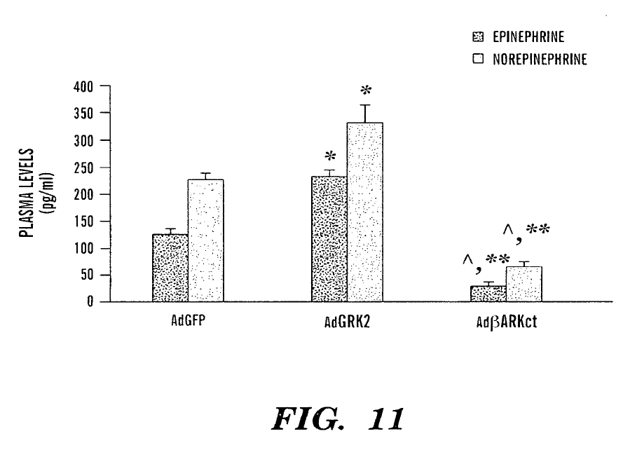

Having established efficacious in vivo adrenal-specific gene delivery via the two different methods described above, the effects of manipulation of adrenal GRK2 levels in vivo on plasma circulating CA levels in normal healthy rats were investigated. As shown in Example 1, GRK2 is a critical regulator of adrenal function in HF. Adrenal glands of three-month-old normal rats were infected in vivo with AdGRK2, AdβARKct, or AdGFP. A separate group of rats received saline treatment as an additional control to assess any potential effects of adenovirus infection and GFP expression. At 7d post-gene delivery, plasma was collected from these rats for determining circulating CA levels. As shown in FIG. 11, plasma levels of both Epi and NEpi in the AdGRK2-treated rats were markedly enhanced compared to control AdGFP-treated rats. Conversely, in rats with adrenal-specific expression of the βARKct (7d after AdβARKct delivery), plasma levels of both CAs were significantly reduced compared to AdGRK2-treated or control AdGFP-treated groups (FIG. 11). The exogenous expression of the transgenes (GRK2 and βARKct) in the adrenal glands of the respective groups was confirmed by western blotting of adrenal protein extracts. Plasma CA levels of saline-treated rats were similar to AdGFP-treated rats. These results confirm that increased adrenal GRK2 activity in vivo leads to elevated circulating plasma CA levels, whereas lowering of adrenal GRK2 activity in vivo has the opposite effect.

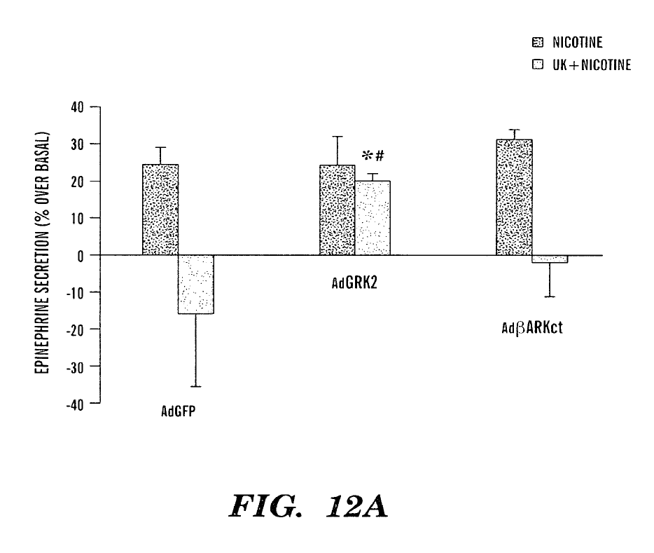

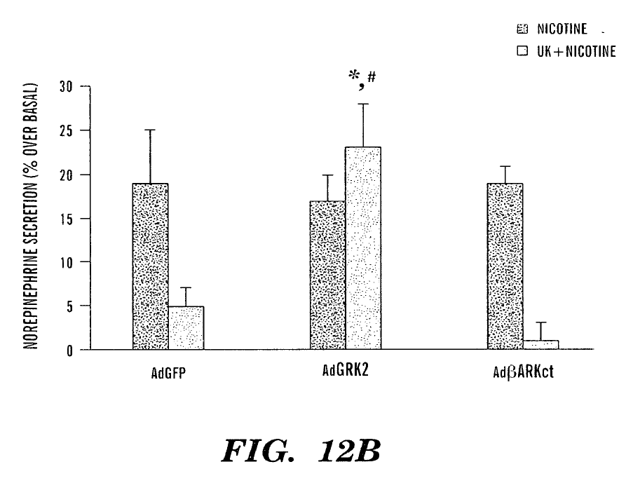

The data for plasma CA levels above suggest that in vivo modulation of adrenal GRK2 activity affects CA secretion from chromaffin cells of the adrenal medulla in normal rats. Because α₂AR-mediated inhibition of CA secretion from these cells is critically regulated by GRK2 as shown in Example 1, the adrenal glands of adenoviral-treated rats (AdGRK2, AdβARKct, AdGFP) were extracted, isolated chromaffin cells and cultured them for 24 hr. Following this period, in vitro CA secretion assays were performed using nicotine to activate nicotinic cholinergic receptors (the physiological stimulus for CA secretion from these cells) and the α₂AR agonist UK14304 to activate these negative feedback receptors. As shown in FIG. 12, nicotine stimulated Epi and NE secretion to an equal extent from the chromaffin cells isolated and cultured from all three groups. Interestingly, in chromaffin cells from control AdGFP-treated rats, UK14304 abolishes CA secretion in response to nicotine as expected, but this effect of UK14304 is lost in chromaffin cells from AdGRK2-treated rats (FIG. 12). In cells from AdβARKct-treated rats, the ability of UK14304 to inhibit CA secretion is normal and similar to control cells treated with AdGFP (FIG. 12). These results indicate that increased adrenal GRK2 levels in vivo can cause enhanced α₂AR desensitization and prevent normal feedback inhibition of CA secretion.

1. A method for treating heart failure in a subject in need thereof comprising administering to the adrenal gland of a subject a vector comprising a nucleic acid sequence encoding a GRK2 antagonist.

2. The method of claim 1 in which said GRK2 antagonist is a βARKct peptide.

3. A method for treating heart failure in a subject in need thereof comprising administering to the subject a vector comprising a nucleic acid sequence encoding a GRK2 antagonist and an adrenal gland targeting moiety.

4. The method of claim 3 in which said GRK2 antagonist is a βARKct peptide.

5. A method for inhibiting the down-regulation and desensitization of peripheral α2-adrenergic receptors (α2-ARs) in a target cell of a subject comprising introducing into said cell a GRK2 inhibitor in an amount and under conditions such that the down-regulation and desensitization of α2-adrenergic receptors is lessened as a result of a decrease of adrenal GRK2 activity thereby reducing the catecholamine release by said target cell of said subject.

6. The method of claim 5, wherein said target cell of said subject is a peripheral sympathetic neuron, a cardiac sympathetic terminal neuron, or a chromaffin cell of the adrenal medulla.

7. The method of claim 5, wherein said GRK2 inhibitor is a βARKct peptide, a GRK2 siRNA, or a GRK2 antisense oligonucleotide.

8. The method of claim 5, wherein said GRK2 inhibitor is administered to said target cell of said subject by means of suprarenal or direct intra-suprarenal injection.

9. A method for treating an endocrinological or a non-endocrinological disease characterized by an elevation of sympathetic nervous system (SNS) activity/outflow and catecholamine turnover in a subject comprising introducing into a target cell of said subject a therapeutically effective amount of a GRK2 inhibitor and a pharmaceutically acceptable carrier.

10. A pharmaceutical composition for treating or preventing chronic heart failure in a subject comprising a therapeutically effective amount of a GRK2 inhibitor suitable for delivery to adrenal cells, wherein said treatment results in a decrease of adrenal GRK2 activity, a decrease in down-regulation and desensitization of α2-adrenergic receptors, and thereby reduces the catecholamine release in said subject.

11. A combined pharmaceutical composition for treating or preventing chronic heart failure comprising a GRK2 inhibitor and an agent selected from the group consisting of β-blocker, angiotensin-converting enzyme (ACE) inhibitor, or angiotensin-renin blocker (ARB), wherein delivery of said composition results in a decrease of adrenal GRK2 activity, a decrease in down-regulation and desensitization of α2-adrenergic receptors, and a reduction of catecholamine in a subject experiencing chronic heart failure.

12. The combined pharmaceutical composition of claim 11, wherein said β-blocker is selected from the group consisting of Sectral® (acebutolol), Zebeta® (bisoprolol), Brevibloc® (esmolol), Inderal® (propanolol), Tenormin® (atenolol), Normodyne®, Trandate® (labetalol), Coreg® (carvedilol), Lopressor®, and Toprol-XL® (metoprolol).

13. The combined pharmaceutical composition of claim 11, wherein said ACE inhibitor is selected from the group consisting of Lotensin® (benazepril), Capoten® (captopril), Vasotec® (enalapril), Monopril® (fosinopril), imidapril, Prinivil® (lisinopril), Zestril®, Univasc® (moexipril), Accupril® (quinapril), Aceon® (perindopril erbumine), Altace® (ramipril), and Mavik® (trandolapril).

14. The combined pharmaceutical composition of claim 11, wherein said angiotension II receptor blocker is selected from the group consisting of Atacand® (Candesartan cilexetil), Teveten® (Eprosartan), Avapro® (Irbesartan), Cozaar® (Losartan) Benicar® (Olmesartan medoxomil), Micardis® (Telmisartan) and Diovan® (Valsartan).
